(19) United States
(12) Patent Application Publication
(10) Pub. No.: US 2025/0116658 A1
(43) Pub. Date: Apr. 10, 2025

(54) COMPOSITIONS AND METHODS FOR IN VIVO PROTEASE PROFILING BY IMMUNE CELL DISPLAY

(71) Applicant: GEORGIA TECH RESEARCH CORPORATION, Atlanta, GA (US)

(72) Inventors: Gabriel A. KWONG, Atlanta, GA (US); Marielena GAMBOA, Atlanta, GA (US); Quoc MAC, Atlanta, GA (US); Hathaichanok PHUENGKHAM, Atlanta, GA (US); Anirudh SIVAKUMAR, Atlanta, GA (US); Ali ZAMAT, Atlanta, GA (US)

(21) Appl. No.: 18/292,706

(22) PCT Filed: Jul. 25, 2022
(86) PCT No.: PCT/US2022/074114

§ 371 Date: Dec. 2, 2024

Related U.S. Application Data
(60) Provisional application No. 63/225,674, filed on Jul. 26, 2021.

(51) Int. Cl. G01N 33/50; C07K 14/705; C07K 14/71; C07K 14/725; C07K 16/32; C12N 5/0783

(52) U.S. Cl. CPC G01N 33/5091; C07K 14/7051; C07K 14/70517; C07K 14/71; C07K 16/32; C12N 5/0636; G01N 33/505; C07K 2317/622; C07K 2319/02; C07K 2319/03; G01N 2333/96436

(57) ABSTRACT

. There is great interest in developing strategies to identify proteolytic substrates. Synthetic peptide libraries are among the most widely used to screen substrates-based activity, however, this technique is limited by the size of libraries and is time-consuming. Several combinatorial library-based display technologies such as phage, bacteria and yeast display, can identify better substrates for protease activity with higher-throughput sequencing and enrichment of specific substrates over multiple rounds of selection. Even though these profiling techniques provide greater coverage of protease cleavable substrates, such cells are less capable of accessing difficult to deliver sites, pose immunogenicity concerns, and have not been applied to discriminate on-from off-target activity in vivo. Disclosed herein are in vitro and in vivo methods for screening protease substrates.

### CROSS-REFERENCE TO RELATED APPLICATIONS

This application is a national stage entry of International Patent Application No. PCT/US2022/074114, filed Jul. 25, 2022, which claims the benefit of priority to U.S. Provisional Application No. 63/225,674, filed Jul. 26, 2021, which are each incorporated by reference herein in their entireties.

### STATEMENT REGARDING FEDERALLY SPONSORED RESEARCH

This invention was made with Government Support under Grant Nos. CA237210 and HD091793 awarded by the National Institutes of Health. The Government has certain rights in the invention.

### BACKGROUND

Dysregulation of the proteolytic balance is often associated with diseases and can signify the disease progression including cancer, autoimmune diseases, inflammation, and other diseases. Proteolysis is an irreversible regulatory mechanism and known to selectively cleave specific substrates. The identification of these peptide substrates has become powerful tools to drive the development of biomarker for early disease detection and new drug design for safer cancer therapies. For example, many proteases are involved in processes upstream of cell death and tissue remodeling that ultimately contribute to disease burden, leading to activity-based strategies for early cancer detection by utilizing cytotoxic protease granzyme B (GzmB) like PET probe, and urinary biomarker. In addition, undesired on-target/off-tumor toxicity of cancer treatments has been associated with most of the clinical responses. Such toxicity motivates several cancer therapies to be designed as prodrugs, probodies, masking cytokines and T-cell bispecifics which are shielded in non-target organ and can be activatable under presence of tumor associated protease. Given the use of protease activity for diagnostic and therapeutic applications, there is need to address the aforementioned problems mentioned above by developing strategies to screen and identify substrates that are selectively cleaved by proteases in the actual pathological site.

### SUMMARY

The present disclosure relates to engineered protease activatable receptors and methods for the manufacture and use thereof.

In one aspect, disclosed herein are engineered protease activatable receptors comprising an extracellular sensor, a transmembrane domain, and an intracellular domain; wherein the extracellular sensor further comprises a masking peptide, a linking peptide, an antigen binding receptor, and a signal transducing complex. In other embodiments, the engineered receptor comprises a chimeric antigen receptor (CAR) or a synthetic Notch (synNotch) receptor.

Also, disclosed are engineered protease activatable receptors of any preceding aspect, wherein the antigen binding receptor is an extracellular surface receptor. In some embodiments, the antigen binding receptor is an anti-human epidermal growth factor 2(αHER2) single-chain variable fragment (scFv) receptor or an anti-epidermal growth factor receptor (αEGFR) scFv receptor. In other embodiments, the antigen binding receptor is a CAR including, but not limited to a CAR targeting CD19, B cell maturation antigen (BCMA), CD22, CD33, CD38, NCAM1, CD5, CD70, MET, Muc1, L1CAM, CD44 SLAMF7, EPHA2, GPC3, mesothelin, or PDCD1), an anti-prostate specific antigen (PSA) receptor, an anti-prostate stem cell antigen (PSCA) receptor, or anti-variable heavy domain of heavy chain (αVHH).

In some aspects, disclosed herein are the engineered protease activatable receptors of any preceding aspects, wherein the masking peptide is 9, 10, 11, 12, 13, 14, 15, 16, 17, 18, 19, 20 or 21 amino acids in length. In one example, in some aspects the masking peptide comprises SEQ ID NO: 1-4. In another example, the masking peptide comprises a HER2 peptide.

Also disclosed herein are the engineered protease activable receptor of any preceding aspects, wherein the linking peptide is 5, 6, 7, 8, 9, 10, 11, 12, 13, 14, 15, 16, 17, 18, 19, 20, 21, 22, 23, 24, or 25 amino acids in length. In some embodiments, the linking peptide comprises a protease cleavable peptide. In other embodiments, the protease cleavable peptide comprises SEQ ID NO: 5-7.

In some aspects, disclosed herein are the engineered protease activable receptors of any preceding aspects, wherein signal transducing complex comprises a CD8α signaling peptide, an extracellular Notch core, and a juxtamembrane Notch core. In some embodiments, the CD8α signaling peptide comprises SEQ ID NO: 8 or SEQ ID NO: 9. In other embodiments, the extracellular Notch core comprises SEQ ID NO: 10 or SEQ ID NO: 11. In some embodiments, wherein the juxtamembrane Notch core comprises SEQ ID NO: 12.

Also disclosed herein are the engineered protease activable receptors of any preceding aspects, wherein the transmembrane domain is a transmembrane Notch core. In some embodiments, the transmembrane Notch comprises SEQ ID NO: 13 or SEQ ID NO: 14.

In some aspects, disclosed herein are the engineered protease activable receptors of any preceding aspects, wherein the intracellular transcription factor activates a reporter gene. In some embodiments, the transcription factor comprises a Gal4-VP64 transcription factor. In some embodiments, the reporter gene comprises luciferase, green fluorescent protein (GFP), yellow fluorescent protein (YFP), blue fluorescent protein (BFP), cyan fluorescent protein (CFP), monomeric red fluorescent protein (mRFP), Discosoma striata (DsRed), mCherry, mOrange, tdTomato, mStrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, EBFP2, herpes simplex virus 1 thymidine kinase (HSV-TK), and/or sodium iodide symporter (NIS).

Disclosed herein are immune cells comprising the engineered protease activatable receptor of any preceding aspects. In some embodiments, the immune cell is a T cell, a macrophage, a dendritic cell, a natural killer (NK) cell, or NK T cell. In some embodiments, the immune cell further comprises a library of protease substrates. In some embodiments, the library comprises a random 3, 4, 5 or 6 amino acid peptides.

Also disclosed herein is an in vitro method of screening an assortment of protease substrates for monitoring dysregulated protease enzymes in a cancer comprising: a. culturing a cancerous sample expressing a target antigen with i) an immune cell comprising an extracellular sensor, a transmembrane domain, and an intracellular transcription factor; wherein the extracellular sensor further comprises a masking peptide, a protease substrate, an antigen binding receptor, and a signal transducing complex and ii) a protease; wherein a substrate that is a specific target for the protease is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene and b. detecting expression of a reporter gene. In some embodiments, the method further compromises identifying the sequence of the protease substrate by sequencing. In other embodiments, the method further comprises successively performing the screen 2, 3, 4, or 5 times and each subsequent time only using the top 10% of sequences from the previous round.

In some aspects, disclosed herein is an in vitro method of screening an assortment of protease substrates for monitoring dysregulated protease enzymes in a cancer of any preceding aspects, wherein any one of the protease substrate library comprises a 3, 4, 5 or 6 random amino acid linker.

Disclosed herein is a method of monitoring a cancer (such as for example, a solid tumor, including, but not limited to an epithelial carcinoma, a sarcoma, a lymphoma, a blastoma, or a melanoma) in a subject comprising administering an engineered cell comprising an extracellular sensor, a transmembrane domain, and an intracellular transcription factor. In some aspects, disclosed herein is a method of monitoring a cancer of any preceding aspect wherein the extracellular sensor further comprises a masking peptide, a linking peptide, an antigen binding receptor, and a signal transducing complex. In some aspects, a protease enzymes cleaves the linking peptide at allow an interaction between the engineered cell and a cancer cell. In some embodiments, the interaction involves binding the antigen binding receptor of the engineered cell to a surface antigen of the cancer cell. In other embodiments, the interaction activates the intracellular transcription factor and expression of a reporter gene. In some aspects, the engineered cell is an engineered immune cell (such as, for example, an engineered T cell, macrophage, dendritic cell, natural killer (NK) cell, or NK T cell).

Also disclosed herein are disclosed herein are in vivo methods of screening of protease substrates for monitoring dysregulated protease enzymes in a cancer (such as for example, a solid tumor, including, but not limited to an epithelial carcinoma, a sarcoma, a lymphoma, a blastoma, or a melanoma) comprising step of a) obtaining an engineered immune cell (such as, for example, an engineered T cell, macrophage, dendritic cell, natural killer (NK) cell, or NK T cell) comprising a protease activatable receptor (CAR or synNotch receptor) which comprises an extracellular sensor, a transmembrane domain, and an intracellular domain; wherein the extracellular sensor comprises a masking peptide, a library of protease substrates and an antigen binding receptor; b) transferring the engineered immune cells to tumor bearing mice expressing target antigen; wherein the tumor microenvironment is protease enriched; wherein a substrate that is a specific target for the proteases is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; wherein binding of the target further causes tumor tissue to be dissociated; and c) detecting expression of the reporter gene disclosed herein are in vivo methods of screening of protease substrates for monitoring dysregulated protease enzymes in a cancer comprising step of a) obtaining an engineered immune cell comprising a protease activatable receptor (CAR or synNotch receptor) which comprises an extracellular sensor, a transmembrane domain, and an intracellular domain; wherein the extracellular sensor comprises a masking peptide, a library of protease substrates and an antigen binding receptor; b) transferring the engineered immune cells to tumor bearing mice expressing target antigen; wherein the tumor microenvironment is protease enriched; wherein a substrate that is a specific target for the proteases is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; wherein binding of the target further causes tumor tissue to be dissociated; and c) detecting expression of the reporter gene. In some aspects, the method can comprise identifying the sequence of the protease by sequencing (including, but not limited to next generation sequencing (NGS).

In some aspects, disclosed herein are in vivo methods of screening of protease substrates for monitoring dysregulated protease enzymes of any preceding aspect, further comprising sorting for the expression of the reporter gene.

Also disclosed herein are in vivo methods of screening of protease substrates for monitoring dysregulated protease enzymes of any preceding aspect, further comprising detecting and sorting cells expressing the reporter gene by negative selection.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows the overall studies to develop T cell display platform to identify peptide substrates in vitro and in vivo. Engineered T cell display present genetically encoded millions of substrate sequences libraries. These T cell display traffic to disease sites in vivo. Engineered T cells express a masking peptide to block the antigen binding site, these linkers are then cleaved by responsive T cell and tumor proteases. Antigen binding then enables selection of protease-activated T cells and subsequent recovery of protease substrates by NGS and data analysis to determine the winner sequence.

FIG. 2 shows the overall approach to investigate masked synNotch T cell display.

FIG. 3A shows the engineered protease-activable T cell display (3A) Schematic of protease-actuated T cell construct and design. The masking peptide blocked antigen-binding site and is cleaved with particular protease to expose antigen binding and then activate the synNotch reporter. FIG. 3B shows flow staining shows HER2 antigen binding (using HER2-biotin+steptravidin-APC) of protease-actuated T cell encoded with Thrb cleavable linker with and without incubation with Thrb or Thrb inhibitor (bivalirudin) for 30 min at 37° C. FIG. 3C shows histogram shows BFP expression of protease-actuated T cell coincubated with MDA-MB-468 expressing HER2 cells with and without incubation with Thrb or bivalirudin for 24 h at 37° C. (****P<0.0001, one-way ANOVA and Tukey post-test and correction, mean±SEM is depicted, n=3 biologically independent wells).

FIGS. 4A and 4B show the heat map summarizing the protease cleavage of define substrates. Comparison of protease sensing activity of (4A) synthetic peptide fluorogenic probe (incubation for 30 min) and (4B) protease-actuated T cells (incubation with protease and MDA-MB-468 HER2+ for 24 h). (****P<0.0001, one-way ANOVA and Tukey post-test and correction, mean±SD is depicted, n=3 biologically independent wells).

FIG. 5 shows the schematic for T cell display library, screening, and NGS and analyzing.

FIGS. 6A and 6B show the generation of 3-mer linker of T cells library (6A) Schematic representation of a protease-activable αEGFR CAR T cell display genetically encoded libraries of 3-mer linker to create a library size of 8,000 amino acid sequences. The generated library was investigated by NGS which show a library size of 3,943 amino acid sequences. (6B) Multiple round screening by T cell display against Thrb protease. The bar graphs showing percentage of reporter activated cell in each round of the screening. (****P<0.0001, one-way ANOVA and Tukey post-test and correction, mean±SD is depicted, n=3).

FIGS. 7A shows the in vitro screening of Thrb substrates (7A) Schematic of a protease-activable αEGFR CAR T genetically encodes libraries of protease-cleavable linkers containing 3-mer random linker screening against Thrb. Activated cells were identified for protease substrate sequences by NGS. FIG. 7B shows the read count frequency of unique sequences found in the screening. FIG. 7C shows the alignment of peptides sequenced generation (Thrb treated sorted cells) of a sequence consensus. FIG. 7D shows the unique sequences from the screening (Δ Freq.>0; LVSPRSG (SEQ ID NO: 20) and Δ Freq.<0; LVSFPSG (SEQ ID NO: 21), LVQNLSG (SEQ ID NO: 22) were validated the cleavage activity in fluorogenic assays.

FIGS. 8 shows the schematic for T cell display library, isolation, sorting, and NGS and analyzing.

FIGS. 9A-9D show the established murine E0771 breast cancer cells expressing human HER2 antigen. FIG. 9A shows a schematic of generation HER2 antigen expressing E0771 breast cancer cells with different HER2 expression level. Histogram shows different expression level of HER2 antigen of each sorted clone stained with αHER2-FITC. FIG. 9B shows different synNotch reporter activation level based on target antigen density. The expression level of BFP reporter upon αHER2 synNotch binding to HER2 antigen on E0771 cancer cells after 24 hours incubation. Data are presented as the means±S.D. (n=3). FIG. 9C shows a tumor growth cure of human HER2 expressing E0711 in B6 vs B6-Tg (WapHER2) mice. FIG. 9D shows a flow plot of human HER2 antigen expressing tumor cell which isolated from E0771-HER2 bearing vs B6-Tg (WapHER2) mice after 21 days of tumor inoculation.

FIG. 10A-10C shows the activation of masked synNotch T cell display in vivo. Ten million of αHER2 masked synNotch genetically encoded libraries of 4-mer linker were intravenously injected to MDA-MB-468 HER+ bearing and healthy NSG mice. (10A) The likers are cleaved by tumor proteases and antigen binding then enables activate BFP reporter. (10B) representative flow plot and (10C) bar graph of percent of BFP+ cell in tumor and other organs at 24 h after adoptive T cell transfer. (****P<0.0001, one-way ANOVA and Tukey post-test and correction, mean±SD is depicted, n=3)

FIGS. 11A-11C show the luciferase-based optical imaging synNotch reporter to detect protease activity. FIG. 11A shows a schematic representation of Gal4 response elements to co-expresses GFP and luciferase construct. FIG. 11B shows luminescence images and (11C) bar graphs showed the synNotch activation and downstream high luciferase expression upon proteolysis. (****P<0.0001, one-way ANOVA and Tukey post-test and correction, mean±SD is depicted, n=3 biologically independent wells).

FIGS. 12A and 12B show the detection of synNotch activation in vivo using luciferase reporter. Engineered T cells with luciferase reporter were transferred into both sites of tumor with one cohort receiving αHer2 synNotch cells and a separate cohort receiving αHer2 masked synNotch cells with GSGGSG linker. FIG. 12A shows luciferase expression kinetics of adoptive cell transfer. FIG. 12B shows representative luciferase image and bar plot of mice after 24 h of the transferring. (*P<0.05, one-way ANOVA and Tukey post-test and correction, mean±D is depicted, n=3).

FIGS. 13A-13E shows the engineered protease-activable CAR T cell display (13A) Schematic of masked CAR T cell display design. The masking peptide blocked antigen-binding site and is cleaved with particular protease to expose antigen binding. (13B) Flow staining and (13C) bar graph on EGFR antigen binding of m.CAR cells with and without incubation with Thrb or Thrb inhibitor (bivalirudin) for 30 min at 37° C. (13D) Flow plot and (13E) bar graph on CD69 expression of m.CAR cells coincubated with MDA-MB-231 expressing EGFR cells with and without incubation with Thrb or bivalirudin. for 24 h at 37° C. (****P<0.0001, one-way ANOVA and Tukey post-test and correction, mean±SEM is depicted, n=3 biologically independent wells).

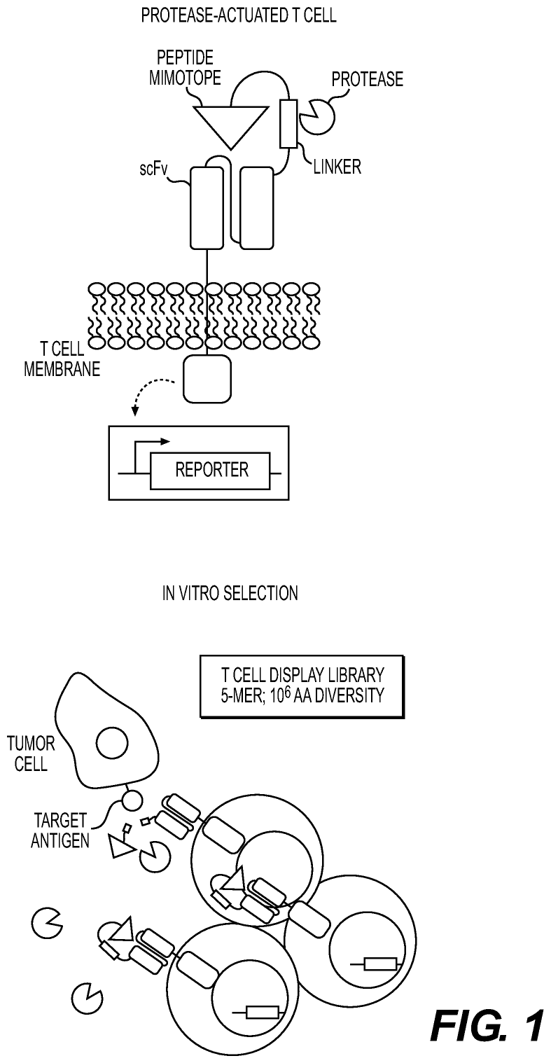

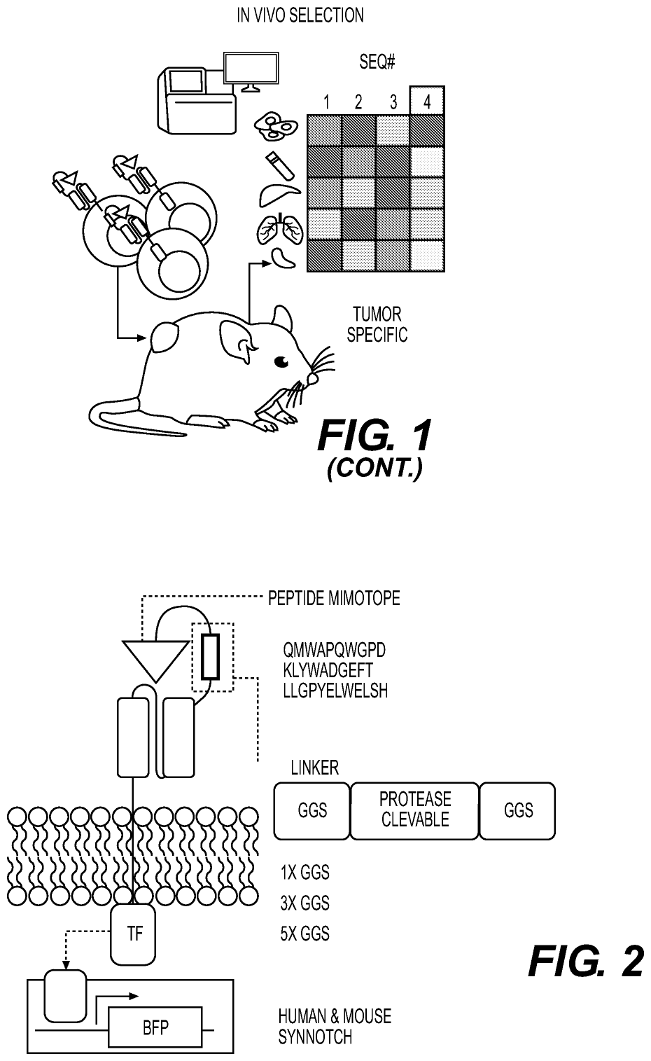

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “transcription factor” refers to a sequence-specific DNA-binding protein that controls the rate of transcription of genetic information from DNA to messenger RNA, by binding to a specific DNA sequence.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art, some of which are described herein.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “detect” or “detecting” refers to the an output signal released for the purpose of sensing of physical phenomenon. An event or change in environment is sensed and signal output released in the form of light.

The term “cancer” is used to address any neoplastic disease and is not limited to epithelial neoplasms (surface and glandular cancers; such a squamous cancers or adenomas)). It is used here to describe both solid tumors and hematologic malignancies, including epithelial (surface and glandular) cancers, soft tissue and bone sarcomas, angiomas, mesothelioma, melanoma, lymphomas, leukemias and myeloma.

The terms “cell,” “cell line” and “cell culture” include progeny. It is also understood that all progenies may not be precisely identical in DNA content, due to deliberate or inadvertent mutations. Variant progeny that has the same function or biological property, as screened for in the originally transformed cell, are included. The “host cells” used in the present disclosure generally are prokaryotic or eukaryotic hosts.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8⁺ “killer” T cells and CD4⁺ “helper T cells.

A “biosensor” or “sensor” refers to a cell, protein, nucleic acid, or biomimetic polymers which can detect analytes or target molecules. These sensors are applied to both in vitro and in vivo applications. These sensors are also encapsulated or confined by means of semipermeable barriers or a polymer matrix to physically or chemically constrain the sensing cell or macromolecule to a scaffold or membrane.

As used herein, “polypeptide” or “peptide” refers to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

“Amino acid” is used herein to refer to a chemical compound with the general formula: NH₂—CRH COOH, where R, the side chain, is H or an organic group. Where R is organic, R can vary and is either polar or nonpolar (i.e., hydrophobic). The following abbreviations are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine. Unless otherwise indicated, the term “amino acid” as used herein also includes amino acid derivatives that nonetheless retain the general formula.

The term “interaction” refers to an action that occurs as two or more objects have an effect on one another either with or without physical contact. In terms of biological interactions, cell, proteins, and other macromolecules can have said effects on one another to impact biological functions, such as cell/tumor growth, cell death, and cell signaling pathways.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

A “mimotope” as used herein refers to a macromolecule, often a peptide, that mimics the structure of an epitope, or a targeted binding site on another protein. Mimotope analyses are commonly used to map epitopes, identify drug targets, and/or uncover protein interacting networks.

“Expression” as used herein refers to the process by which information from a gene is used in the synthesis of a functional gene product that enables it to produce a peptide/protein end product, and ultimately affect a phenotype, as the final effect.

“Proteolysis” refers to the breakdown of proteins or polypeptides into smaller polypeptides or amino acids. This process, if uncatalyzed, can be extremely slow, taking hundreds of years. Proteolysis typically is a catalyzed process enforced by cellular enzymes called proteases. This also encompasses the term(s) “cleave”, “cleavage”, and “cleavable”, which all refer to breaking of peptide bonds or bonds between amino acids.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

Disclosed herein are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular protease sensor or engineered T cell is disclosed and discussed and a number of modifications that can be made to a number of molecules including the protease sensor or engineered T cell are discussed, specifically contemplated is each and every combination and permutation of the protease sensor or engineered T cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

Strategies to development biomarkers of early disease detection and drug design are emerging in efforts to produce safer cancer therapies. For example, identification of peptide substrates of protease enzymes presents as powerful tool to detect cancer progression in the early stages. Proteolysis, or degradation of proteins, is an irreversible mechanism performed by protease enzymes present in all cell types that cleave specific peptide substrates. In disease, these mechanisms are dysregulated leading to abnormal and/excessive degradation of proteins. In addition, dysregulation of proteolytic mechanisms can upregulate cell death and tissue remodeling pathways further contributing to cancer progression. Current cancer treatments cause on-target/off-tumor toxicity in tumors leading to the development of prodrugs, probodies, masking cytokines, and T-cell bispecifics that shield non-target organs while targeting tumor sites after activation under the presence of tumor-specific proteases. Collectively, these efforts illustrate the need to develop strategies to screen and identify tumor-specific protease substrates to be used to target and prevent protease dysregulation at the tumor site.

Thus, in one aspect, disclosed herein is an engineered protease activable receptor comprising an extracellular sensor, a transmembrane domain, and an intracellular transcription factor. Also disclosed herein is the extracellular sensor further comprising a masking peptide, a linking peptide, an antigen binding receptor, and a signal transducing complex.

In some embodiments, the antigen binding receptor is an extracellular surface receptor. In some embodiments, the antigen binding receptor is either an anti-human epidermal growth factor receptor 2 (αHER2) scFv receptor or an anti-epidermal growth factor receptor (αEGFR) scFv receptor. In other embodiments, engineered receptor comprises either a chimeric antigen receptor (CAR) or a synthetic Notch (synNotch) receptor. In other embodiments, the CAR includes, but is not limited to including, but not limited to a CAR targeting CD19, B cell maturation antigen (BCMA), CD22, CD33, CD38, NCAM1, CD5, CD70, MET, Muc1, L1CAM, CD44 SLAMF7, EGFR, EPHA2, GPC3, HER2, mesothelin, or PDCD1. In some embodiments, the engineered receptor comprises an anti-prostate specific antigen (PSA) receptor, an anti-prostate stem cell antigen (PSCA) receptor, or an anti-variable heavy domain of heavy chain (αVHH).

The extracellular sensor is an outer cellular membrane protein comprising a masking peptide, linking peptide, antigen binding receptor, and signal transducing complex. The masking peptide, or mimotope, is a synthetic peptide designed in such a way to limit or prevent binding of a substrate to a specific binding site of a cellular membrane or another protein. The masking peptide can bind and block the binding site to prevent interaction between the substrate and its target. One example of masking peptides are human epidermal growth factor receptor (HER2) masking peptides with SEQ ID NO: 1-3. In some embodiments, the masking peptide is 9, 10, 11, 12, 13, 14, 15, 16, 17, 18, 19, 20 or 21 amino acids long. Examples of sequences for one or more masking peptides are provided in Table 1. In some embodiments, the masking peptide comprises or consists of amino acid sequences of any one of SEQ ID NO: 1-4.

TABLE 1
Masking peptide/Mimotope Sequences

| | | SEQ |
|---|---|---|
| | | ID |
| Description | Sequence | NO: |
| 10-mer HER2 peptide | QMWAPQWGPD | 1 |
| mimotope | | |
| 10-mer HER2 peptide | KLYWADGEFT | 2 |
| mimotope | | |
| 12-mer HER2 peptide | LLGPYELWEL | 3 |
| mimotope | SH | |
| 21-mer EGFR masked | QGQSGQCISP | 4 |
| peptide mimotope | RGCPDGPYVM | |
| | Y | |

Linking peptides are peptides comprising protease cleavable peptide flanked by glycine-glycine-serine (GGS) spacer peptides. In some embodiments, the protease cleavable peptides comprise amino acid sequences to protease substrates of Granzyme B (GzmB), Thrombin (Thrb), or matrix metalloproteinase 9 (MMP9). In some embodiments, the protease cleavable peptide comprises amino acid sequences SEQ ID NO: 5-7 as shown in Table 2. In other embodiments, the linking peptide comprises a 3 amino acid (3-mer), a 4 amino acid (4-mer), a 5 amino acid (5-mer), or a 6 amino acid (6-mer) protease cleavable peptide wherein each residue of the 3-mer, 4-mer, 5-mer, or 6-mer can comprise any one of 20 amino acids. In some embodiments, synthetic DNA containing NNK degenerate codons are used to create libraries of linking peptides. An example of the synthetic DNA is CTTGTANNKNNKNNKGTGGGG (SEQ ID NO: 23), wherein N=adenine, guanine, cytosine, or thymine; K=thymine or guanine. In other embodiments, the GGS spacer peptides can be repeated 1, 2, 3, 4, or 5 times. In some embodiments, the spacer peptide is GGSGGSGGS (SEQ ID NO: 17) or GGSGGSGGSGGSGGS (SEQ ID NO: 18) with the GSGGSG spacer peptide (SEQ ID NO: 15) as a negative control. One example of a linking peptide is GGS-XXXXX-GGS (SEQ ID NO: 37). In some embodiments, X=any one of 20 amino acids. In some embodiments, linking peptide is 5, 6, 7, 8, 9, 10, 11, 12, 13, 14, 15, 16, 17, 18, 19, 20, 21, 22, 23, 24, or 25 amino acids in length. In other embodiments, thrombin (Thrb) is the target protease, for which candidate substrate peptide sequences have been generated to create Thrb specific linker peptides using the generic peptide sequence with SEQ ID NO: 19. In some embodiments, the candidate Thrb substrate peptide sequences are shown in Table 3.

TABLE 2
Protease cleavable peptide sequences

| | | SEQ |
|---|---|---|
| | | ID |
| Description | Sequence | NO: |
| GzmB protease cleavable | IEFDSG | 5 |
| linker | | |
| Thrb protease cleavable | LVPRGSG | 6 |
| linker | | |
| MMP9 protease cleavable | PLGLAG | 7 |
| linker | | |

TABLE 3
Thrombin (Thrb) substrate peptide sequences

| | | SEQ |
|---|---|---|
| | | ID |
| Description | Sequence | NO: |
| Generic Thrb substrate | LVXXXSG | 19 |
| peptide | | |
| Thrb substrate peptide | LVSPRSG | 20 |
| (candidate 1) | | |
| Thrb substrate peptide | LVSFPSG | 21 |
| (candidate 2) | | |
| Thrb substrate peptide | LVQNLSG | 22 |
| (candidate 3) | | |
| Thrb substrate peptide | LVPRGSG | 6 |
| (candidate 4) | | |

The signal transducing complex is comprised of multiple proteins embedded or associated with the cellular membrane that relays the protease cleaving activity occurring outside the cell to intracellular compartments. In some embodiments, the signal transducing complex comprises a CD8α signaling peptide with SEQ ID NO: 8 or SEQ ID NO: 9. In some embodiments, the signal transducing complex also comprises a Notch Core further comprising an extracellular domain, ILDYSFTGGAGRDIPPPQIEEACELPECQVDAGNKVCNLQCNNHACGWDGGDCSLN FNDPWKNCTQSLQCWKYFSDGHCDSQCNSAGCLFDGFDCQLTEGQCNPLYDQYCK DHFSDGHCDQGCNSAECEWDGLDCAEHVPERLAAGTL VL VVLLPPDQLRNNSFHFL RELSHVLHTNVVFKRDAQGQQMIFPYYGHEEELRKHPIKRSTVGWATSSLLPGTSGG RQRRELDPMDIRGSIVYLEIDNRQCVQSSSQCFQSATDVAAFLGALASLGSLNIPYKI EAVKSEPVEPPLPSQ (SEQ ID NO: 10) or CPRAACQAKRGDQRCDRECNSPGCGWDGGDCSLSVGDPWRQCEALQCWRLFNNS RCDPACSSPACLYDNFDCHAGGRERTCNPVYEKYCADHFADGRCDQGCNTEECGW DGLDCASEVPALLARGVLVLTVLLPPEELLRSSADFLQRLSAILRTSLRFRLDAHGQA MVFPYHRPSPGSEPRARRELAPEVIGSVVMLEIDNRLCLQSPENDHCFPDAQSAADY LGALSAVERLDFPYPLRDVRGE (SEQ ID NO: 11), a transmembrane domain with SEQ ID NO: 13 or SEQ ID NO: 14, and a juxtamembrane domain with SEQ ID NO: 12. The peptide sequences for the components of the signal transducing complex are shown in Table 4. The signal transducing complex is bound by an intracellular transcription factor. Upon proteolytic cleavage of the linking peptide, the transcription factor travels to the nucleus to activate a reporter. In some, embodiments, the transcription factor is Gal4-VP64. In other embodiments, the transcription factor is tetR-VP64(tTA). In some embodiments, the reporter is a blue fluorescent protein (BFP). In other embodiments, the reporter is a luciferase, green fluorescent protein (GFP), yellow fluorescent protein (YFP), cyan fluorescent protein (CFP), monomeric red fluorescent protein (mRFP), Discosoma striata (DsRed), mCherry, mOrange, tdTomato, mStrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photo switchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2,mOrange2, mApple, Sirius, Azurite, EBFP, EBFP2, herpes simplex virus 1 thymidine kinase (HSV-TK), or sodium iodide symporter (NIS).

TABLE 4
Signal transducing complex peptide sequences

| | | SEQ |
|---|---|---|
| | | ID |
| Description | Sequence | NO: |
| CD8α signaling peptide | MASPLTRFLSLNLL | 8 |
| (mouse) | LLGESIILGSGEA | |
| CD8α signaling peptide | MALPVTALLLPLAL | 9 |
| (human) | LLHAARP | |
| Juxtamembrane Notch | RKRRR | 12 |
| core domain | | |
| Transmembrane Notch | LMYVAAAAFVLLF | 13 |
| core domain (mouse) | FVGCGVLLS | |
| Transmembrane Notch | LPLLVAGAVLLLV | 14 |
| core domain (human) | ILVLGVMVA | |

In another aspect, disclosed herein is an immune cell comprising the engineered protease activatable receptor disclosed above. In some embodiments, the immune cell is a T cell, a macrophage, a dendritic cell, a natural killer cell, or a natural killer T cell. In other embodiments, the immune cell comprises a library of protease substrates. In other embodiments, the library comprises a random 3-mer, 4-mer, 5-mer, or 6-mer amino acid peptide wherein each residue of the 3-mer, 4-mer, 5-mer, or 6-mer can comprise any one of 20 amino acids.

### Methods of Treatment

The disclosed protease activable receptors and immune cells (i.e., recombinant T cell receptor (TCR) T cells, CAR T cells, macrophages, dendritic cells, NK cells, and/or NK T cells) comprising said protease activable receptors can be used to treat any disease, including, but not limited to cancers, with uncontrolled protease activity. A representative but non-limiting list of cancers that the disclosed compositions can be used to treat is the following: sarcomas, blastomas, lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

Accordingly, disclosed herein are methods of identifying, monitoring, and/or screening a cancer and/or metastasis (such as for example, a solid tumor including, but not limited to, epithelial carcinoma, a sarcoma, a lymphoma, a blastoma, or a melanoma) in a subject comprising screening an assortment of protease substrates for monitoring dysregulated protease enzymes in a cancer comprising: a. culturing a cancerous sample expressing a target antigen with i) an immune cell comprising an extracellular sensor, a transmembrane domain, and an intracellular transcription factor; wherein the extracellular sensor further comprises a masking peptide, a protease substrate, an antigen binding receptor, and a signal transducing complex and ii) a protease; wherein a substrate that is a specific target or the protease is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene and b) detecting expression of a reporter gene. In some embodiments, the method further compromises identifying the sequence of the protease substrate by sequencing. In some embodiments, the method further compromises successively performing the screen at least twice, thrice, 4, 5, 6, 7, 8 9, or 10 times, including, but not limited to 2, 3, 4, 5, 6, 7, 8, 9, 10, 11, 12, 13, 14, 15, 16, 17, 18, 19, 20, 21, 22, 23, 24, or 25 times, and each subsequent time only using the top 10% of sequences from the previous round. In other embodiments, any one of protease substrate previously disclosed comprises a 3, 4, 5, or 6 amino acid linker peptides.

It is understood and herein contemplated that screening for protease substrates can also occur in vivo. Thus, disclosed herein are disclosed herein screening of protease substrates for monitoring dysregulated protease enzymes are in vivo methods of screening of protease substrates for monitoring dysregulated protease enzymes in a cancer (such as for example, a solid tumor, including, but not limited to an epithelial carcinoma, a sarcoma, a lymphoma, a blastoma, or a melanoma) comprising step of a) obtaining an engineered immune cell (such as, for example, an engineered T cell, macrophage, dendritic cell, natural killer (NK) cell, or NK

T cell) comprising a protease activatable receptor (CAR or synNotch receptor) which comprises an extracellular sensor, a transmembrane domain, and an intracellular domain; wherein the extracellular sensor comprises a masking peptide, a library of protease substrates and an antigen binding receptor; b) transferring the engineered immune cells to tumor bearing mice expressing target antigen; wherein the tumor microenvironment is protease enriched;

wherein a substrate that is a specific target for the proteases is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; wherein binding of the target further causes tumor tissue to be dissociated; and c) detecting expression of the reporter gene disclosed herein are in vivo methods of screening of protease substrates for monitoring dysregulated protease enzymes in a cancer comprising step of a) obtaining an engineered immune cell comprising a protease activatable receptor (CAR or synNotch receptor) which comprises an extracellular sensor, a transmembrane domain, and an intracellular domain; wherein the extracellular sensor comprises a masking peptide, a library of protease substrates and an antigen binding receptor; b) transferring the engineered immune cells to tumor bearing mice expressing target antigen; wherein the tumor microenvironment is protease enriched; wherein a substrate that is a specific target for the proteases is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; wherein binding of the target further causes tumor tissue to be dissociated; and c) detecting expression of the reporter gene. In some aspects, the method can comprise identifying the sequence of the protease by sequencing (including, but not limited to next generation sequencing (NGS).

Expression of the reporter gene allows detection of screening of protease substrates for monitoring dysregulated protease enzymes can be performed by any method known in the art. In one aspect, the method can comprise sorting for the expression of the reporter gene.

Also, disclosed herein are methods of identifying, monitoring, and/or screening a cancer and/or metastasis (such as for example, a solid tumor including, but not limited to, epithelial carcinoma, a sarcoma, a lymphoma, a blastoma, or a melanoma) in a subject comprising an extracellular sensor, a transmembrane domain, and an intracellular transcription factor. In some embodiments, the extracellular sensor further compromises a masking peptide, a linking peptide, an antigen binding receptor, and a signal transducing complex. In some embodiments, a protease enzyme cleaves the linking peptide to allow an interaction between the engineered cell and a cancer cell. In some embodiments, the interaction involves binding the antigen binding receptor of the engineered cell to a surface antigen of the cancer cell. In some embodiments, the interaction activates the intracellular transcription factor and expression of a reporter gene including, but not limited to blue fluorescent protein (BFP), luciferase, green fluorescent protein (GFP), yellow fluorescent protein (YFP), cyan fluorescent protein (CFP), monomeric red fluorescent protein (mRFP), Discosoma striata (DsRed), mCherry, mOrange, tdTomato, mStrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, EBFP2, herpes simplex virus 1 thymidine kinase (HSV-TK), and/or sodium iodide symporter (NIS). In some embodiments, the engineered cell is an engineered immune cell. In some embodiments, cancer is a solid tumor including, but not limited to, epithelial carcinoma, a sarcoma, a lymphoma, a blastoma, or a melanoma.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: In Vivo Protease Profiling by Immune Cell Display

Protease-actuated synNotch. SynNotch receptor is a powerful tool for engineering cell circuitry for programmed multicellular morphologies, localized tumor control, multi-antigen tumor recognition, and tumor antigen density discrimination. These are the first studies to demonstrate masked synNotch T cell to control synNotch activation by protease activity in biological environment for detection of cancer.

T cell display platform as protease sensors. Here, extensive expertise is used to design activity-based nano sensors to detect and monitor disease as well as engineer T cells to remotely control the antitumor activity. This example integrates both areas by utilizing an engineered T cell as a display platform to screen protease activity. These T cell displays can genetically encode libraries containing millions of peptides and also efficiently deliver the libraries to disease sites.

Screening in actual in vivo biological environment. Although previously screening methods have their advantages, but none identify protease activity substrates in actual in vivo disease condition. Due to the coexistence of a large number of proteases and their diverse roles in many biological pathways, it is difficult to identify specific cleavage products in a pool of complex cellular components. Conditional or transient protease-substrate interactions may also lead to false-negative discovery of substrates. (e.g., presence of non-specific serum proteases and endogenous protease inhibitor). Here, T cell display platforms are engineered to infiltrate to disease sites and can do on-target/off-target selection without immunogenicity after autologous transfer.

### Example 2: Methods

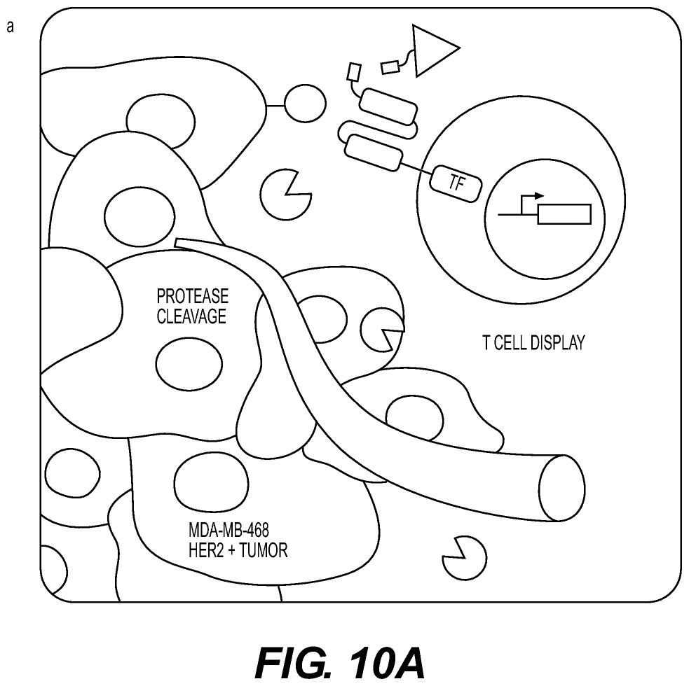

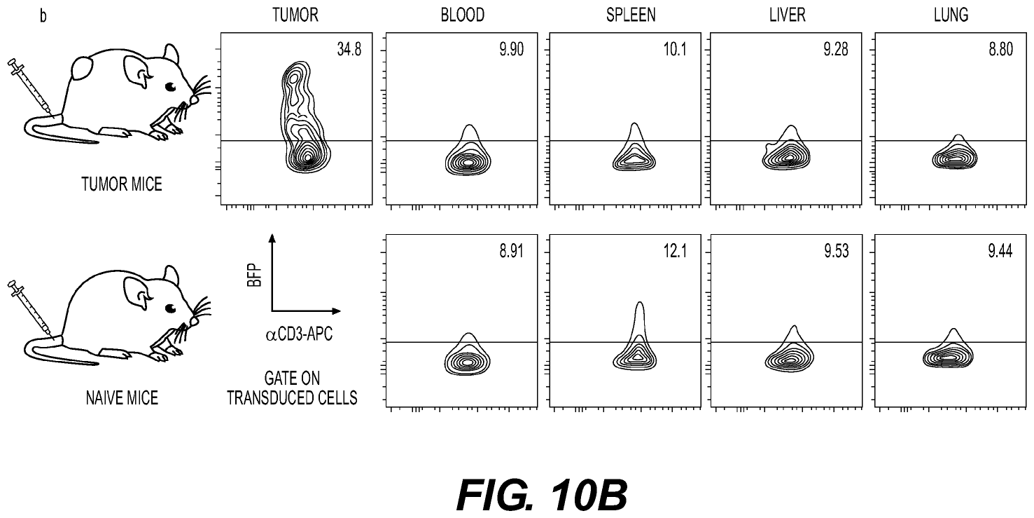

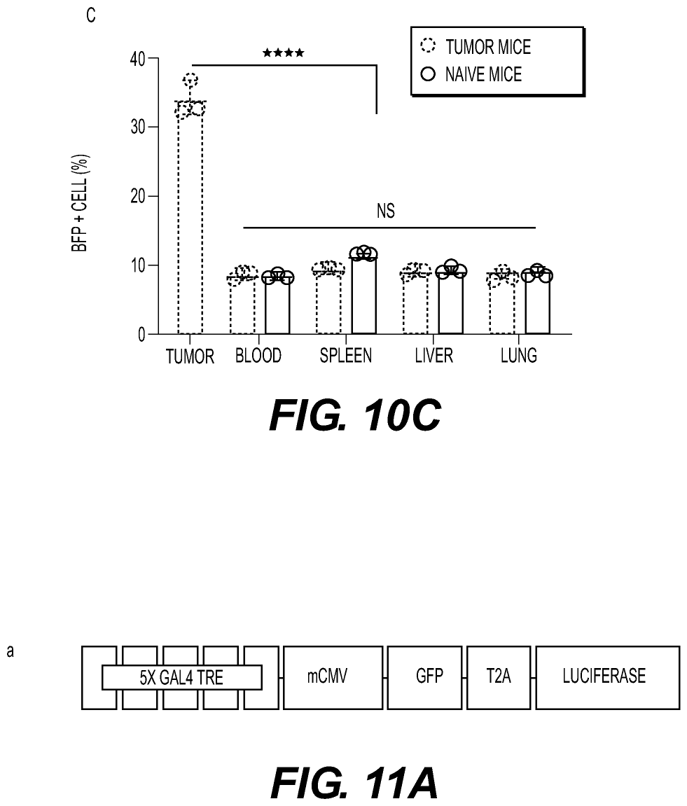

The results herein are rigorous because they achieved statistical difference. Proper control groups were included for comparison, were validated by several metrics, and were reproduced. The screening experiment was (FIG. 10) performed in subcutaneous tumor model. In addition, preliminary biodistribution of T cell display in vivo was conducted in NSG mice lacking an intact immune system. Therefore, this example uses both syngeneic and xenograft models of orthotopic breast tumors by inoculating tumor into mouse mammary fat pad which allows assessment of tumor development in a relevant environment and mimics the disease process in humans.

Rigors of the studies and sex as a biological variable. Rigorous experiment design was achieved by validation of T cell display library (by NGS) and peptide substrate (by fluorogenic probe and luciferase reporter); use of multiple replicates and mouse model; testing of this approach in studies with increasing complexity; extensive experience with sample and animal group size; statistical analyses; multiple levels of controls; and intrinsically paired experimental designs where suitable. As breast cancer is 100 times more common in women than men, female mice were used for these studies. Other factors were considered and justified, such as selection of age, strain, and other underlying condition of animals and biological variables, to ensure experimental rigor.

### Example 3: Masked Synthetic Notch Receptors in Primary T Cells

Genetically encoded display platforms including phage, bacteria and yeast display, have limited use for protease activity screening in vivo due to lack of access to disease sites, immunogenicity concerns, and non-discriminatory on-from off-targeting activity in vivo. To overcome these limitations, a protease-actuated T cell is developed as a T cell sensor to detect proteolytic activity. The engineered T cell consists of synNotch receptor whose binding and activation is blocked by a masking peptide mimotope that is linked by a protease-cleavable motif. Various lengths of the peptide flanking linker are investigated to generate high signal-to-noise for protease sensing T cell display platform (FIG. 2). Both mouse and human-derived synNotch are benchmarked for protease activity screening in syngeneic and xenograft mouse models, respectively.

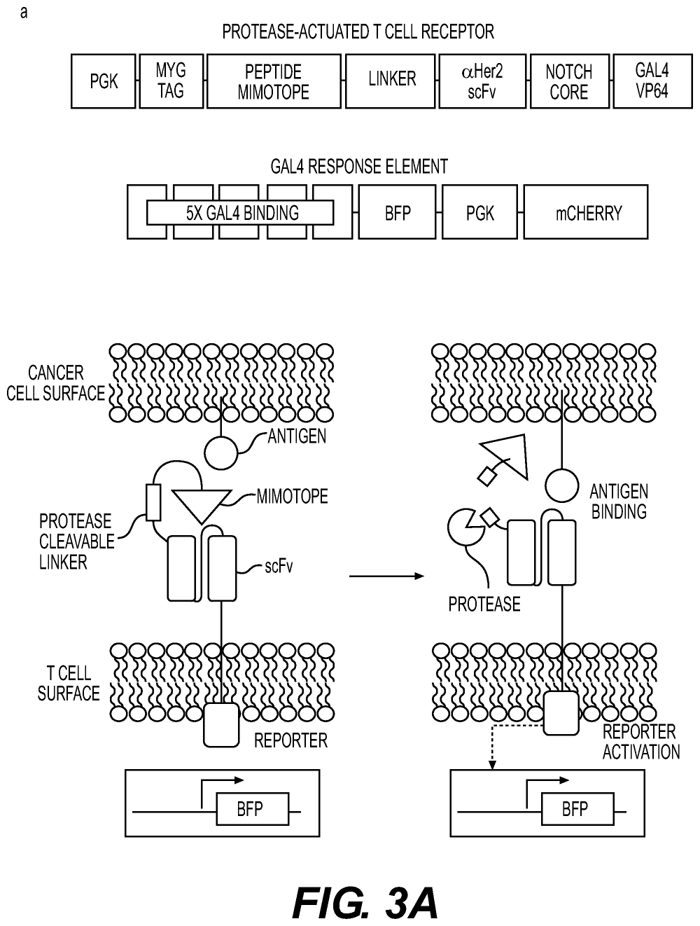

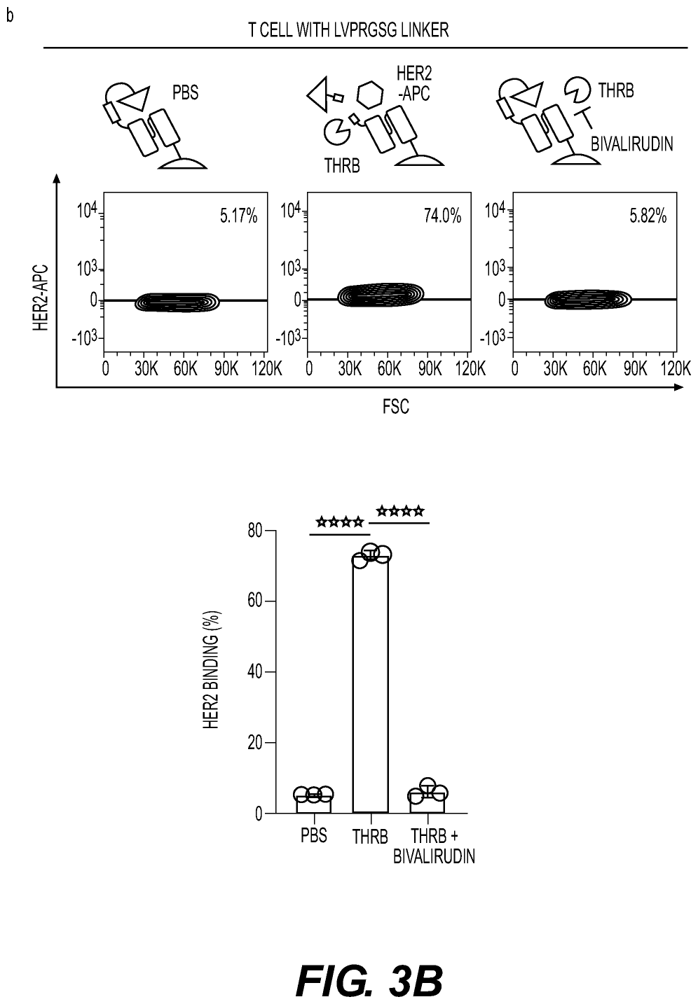

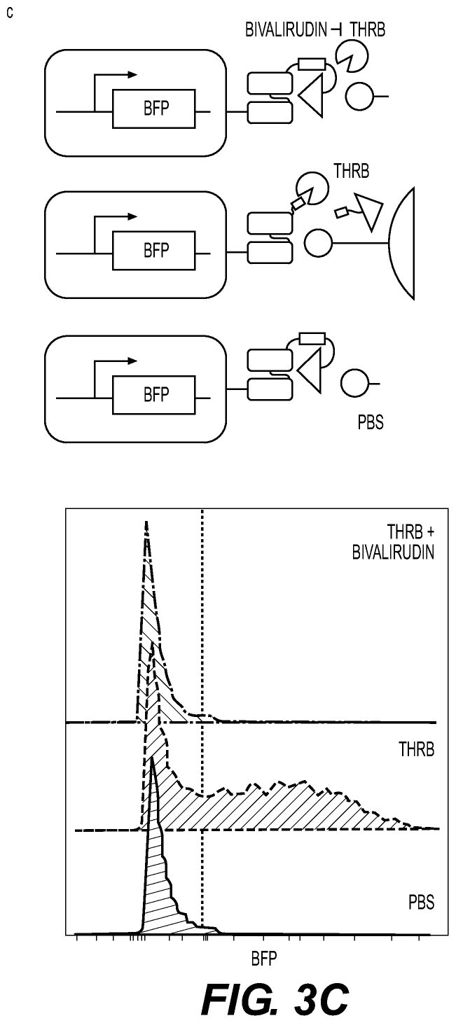

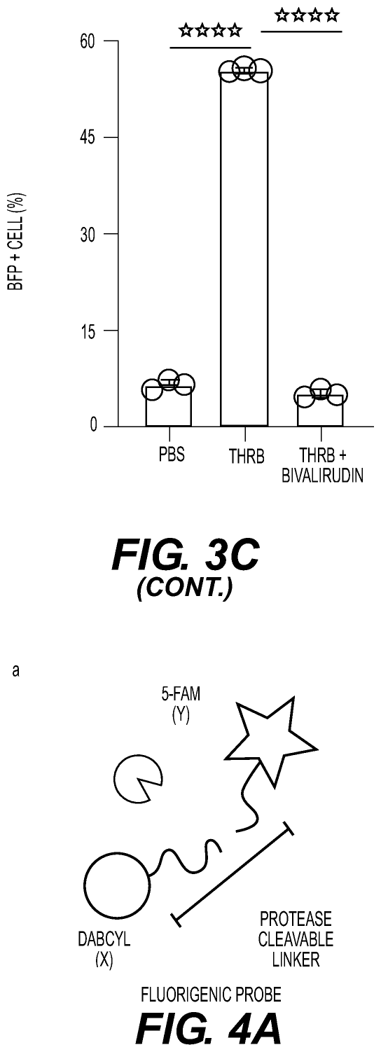

Protease-actuated T cells to sense Thrb protease. T cells engineered with synNotch receptors have robust and highly controlled custom behaviors. Thus, a synNotch response program can be leveraged as protease sensors to allow the creation of a T cell display platform for the selection of specific protease substrates. An anti-human epidermal growth factor receptor 2 (αHER2) masked synNotch was designed, which comprises of a peptide that blocks the antigen-binding site (αHER2 scFv derived from trastuzumab, clone 4D5-7), a protease-sensitive linker, a core regulatory region of Notch, and a cytoplasmic orthogonal transcription factor (TF), Gal4 VP64 controlling expression of BFP, fluorescence reporter gene (FIG. 3a). The masking peptide mimotope, LLGPYELWELSH (SEQ ID NO: 3), derived from a diverse bacterial peptide display library was designed to specifically recognize and block binding of trastuzumab. To investigate the masked synNotch design, LVPRGSG (SEQ ID NO: 6) peptide sequence was selected as Thrombin (Thrb) cleavable linker to link with peptide mimotope and block synNotch activation. The masking peptide was able to effectively block the antigen recognition capability of αHER2 masked synNotchs, however, the binding of the αHER2 scFv receptor to HER2 antigen was largely restored by cleavage of the Thrb-cleavable linker and was still blocked by adding Thrb inhibitor, bivalirudin (FIG. 3b). Considering that the masking peptide diminished the antigen binding of αHER2 scFv to HER2 antigen, the masking peptide also prevented the activation of synNotch reporter toward cocultured MDA-MB-468 breast cancer cell line expressing HER2 antigen in the absence of Thrb, and then subsequently displayed high BFP fluorescence expression upon proteolysis by Thrb and binding stimulation by HER2⁺ cells (FIG. 3c).

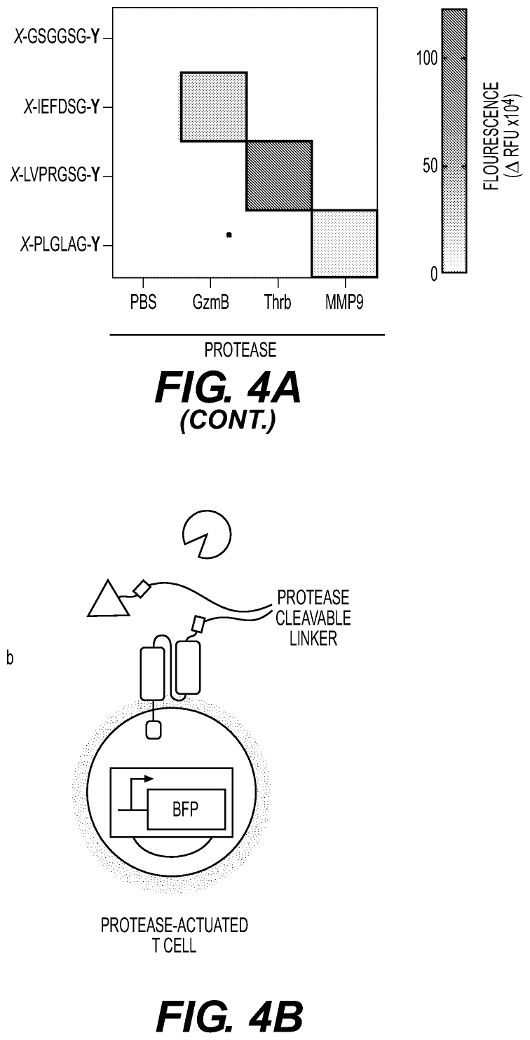

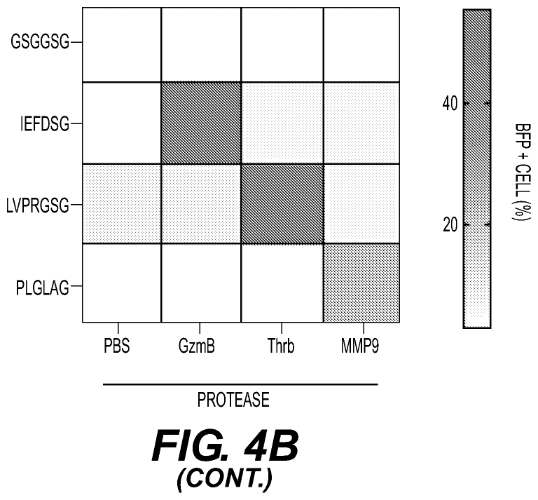

Comparable protease sensing activity of protease cleavable fluorogenic probe and masked synNotch. The masked synNotch was then assessed with identified substrates to ensure each substrate was cleaved by specifically chosen proteases. These masked cells were used to sense the proteases GzmB, matrix metallopeptidase 9 (MMP9) and Thrb which have pathogenic roles in immune responses, cancer, and thrombosis, respectively. These proteases are orthogonal to each other. In addition, a GS-rich linker (GSGGSG (SEQ ID NO: 15)) was designed as a negative control. With orthogonal sequences of protease cleavable linkers for GzmB (IEFDSG (SEQ ID NO. 5)), MMP9 (PLGLAG (SEQ ID NO. 7)), and Thrb (LVPRGSG (SEQ ID NO. 6)), specific cleavage was shown by only the corresponding proteases (FIG. 4a). The masked synNotch design provided comparable reporter activation (FIG. 4b) with the synthetic peptide fluorogenic probe which was commonly used for protease substrate screening in vitro. Taken together, these data show that the masked synNotch was selectively activated in a protease-activated environment and utilized for T cell display platform for protease substrate selection.

Linker length investigation to increase accessibility of proteases. A key element of the success of any screening strategy is the reduction of noise and preservation of signal. To promote efficient antigen screening, the additional flank linker sequence is investigated to provide further flexibility and stability. The effect of incorporating spacers of varying length between target sequence is investigated to increase accessibility of proteases. These include different repeated GGS length (GGS, GGSGGSGGS (SEQ ID NO: 17, GGSGGSGGSGGSGGS (SEQ ID NO: 18)). To quantify protease activity, masked synNotch is cloned with these different GGS spacer length and transduce to T cell. These masked synNotch cells determine the catalytic efficiency by treating them with various concentrations of protease and incubation time. Lastly, off-target cutting is compared using proteases that are found at high levels in circulating blood (e.g., thrombin, plasmin, C1s) to quantify potential background activity.

Blocking efficiency of HER2 peptide mimotope to mask αHER2 scFv. Several HER-2 mimotopes have been identified through phage display (Table 5.) Riemer et al. used a constrained 10-mer random peptide phage display library to identify peptide mimotopes to trastuzumab. Jiang et al. identified another mimotope that matched to an epitope between loops 1 and 2 of HER-2 at the HER-2/trastuzumab interface. Masked synNotch is cloned with these 3 different HER2 mimotopes as shown in Table 5. with a non-cleavable GS-rich linker into a lentiviral vector for delivery to human T cells and is tested for blocking ability of αHER2 scFv to bind to HER2 antigen. A conventional unmasked antigen receptor without the masking peptide and linker sequence is constructed as a control. To assess the decrease of binding capacity of different peptide mimotope-αHER2 scFv toward their target antigen, αHER2 masked synNotch T cell is stained with either protein L or recombinant HER2-Fc fusion protein. Blocking efficiency against varying αHER2 scFv binding affinity is also accessed (4D5-3, 4D5-5, 4D5-7 and 4D5-8 scFv with the order of increasing binding affinity). Lastly, the stability of these peptide mimotopes is evaluated with common proteases that are found at high level in circulating blood (e.g., thrombin, plasmin, C1s) and immunogenicity of these peptide mimotopes is checked by subcutaneously immunize to B6 mice.

TABLE 5
HER2 Peptide mimotope list

| Mimotope Sequence | Length |
|---|---|
| QMWAPQWGPD (SEQ ID NO: 1) | 10-mer |
| KLYWADGEFT (SEQ ID NO: 2) | 10-mer |
| LLGPYELWELSH (SEQ ID NO: 3) | 12-mer |

Mouse vs. human notch signaling components. To utilize the protease activity screening in both syngeneic and xenograft mouse models, masked synNotch is developed for both mouse and human-derived notch. The original synNotch receptor has known limitations that affect its further advancement to clinical translation. These issues include the use of unmatching of species components that could elicit immune rejection. Here, masked synNotch is developed based on murine Notch1 and human Notch3. A masked synNotch against HER2 is built that contained the masking peptide mimotope, protease cleavable linker (use for library screening), the mouse or human Notch core and the Gal4-VP64 TF (Table 6), combining it with the cognate Gal4 DNA response element controlling a BFP reporter. These masked synNotch are tested for protease-dependent T cell activation in co-cultures with recombinant proteases and HER2-positive breast cancer cell lines. The number of target antigen cells and protease concentration are varied. In addition, HER2-negative cancer cells are used as a negative control for demonstration of ligand-dependent signaling.

TABLE 6
Compare synNotch receptor components mouse vs human

| | Mouse | Human |
|---|---|---|
| CD8α signaling | MASPLTRFLSLNLLLLGESIILG | MALPVTALLLPLALLLHAAR |
| peptide | SGEA (SEQ ID NO: 8) | P (SEQ ID NO: 9) |
| Masking | LLGPYELWELSH (or in Table 1) | |
| peptide | (SEQ ID NO: 3) | |
| mimotope | | |
| Protease | GGS-XXXXX-GGS | |
| cleavable linker | (SEQ ID NO: 37) | |
| Antigen binding | HER2 scFv (4D5-7 clone) | |
| Notch core | ILDYSFTGGAGRDIPPPQIEEA | CPRAACQAKRGDQRCDREC |
| (extracellular | CELPECQVDAGNKVCNLQCNN | NSPGCGWDGGDCSLSVGDP |
| domain) | HACGWDGGDCSLNFNDPWKNC | WRQCEALQCWRLFNNSRCD |
| | TQSLQCWKYFSDGHCDSQCNS | PACSSPACLYDNFDCHAGGR |
| | AGCLFDGFDCQLTEGQCNPLY | ERTCNPVYEKYCADHFADGR |
| | DQYCKDHFSDGHCDQGCNSAE | CDQGCNTEECGWDGLDCASE |
| | CEWDGLDCAEHVPERLAAGTL | VPALLARGVLVLTVLLPPEE |
| | VLVVLLPPDQLRNNSFHFLRE | LLRSSADFLQRLSAILRTSL |
| | LSHVLHTNVVFKRDAQGQQMI | RFRLDAHGQAMVFPYHRPSP |
| | FPYYGHEEELRKHPIKRSTVG | GSEPRARRELAPEVIGSVVM |
| | WATSSLLPGTSGGRQRRELDP | LEIDNRLCLQSPENDHCFPD |
| | MDIRGSIVYLEIDNRQCVQSS | AQSAADYLGALSAVERLDFP |
| | SQCFQSATDVAAFLGALASLG | YPLRDVRGE |
| | SLNIPYKIEAVKSEPVEPPLP | (SEQ ID NO: 11) |
| | SQ (SEQ ID NO: 10) | |
| Notch core | LMYVAAAAFVLLFFVGCGVLL | LPLLVAGAVLLLVILVLGVM |
| (transmembrane | S (SEQ ID NO: 13) | VA (SEQ ID NO: 14) |
| domain) | | |
| Notch core | RKRRR (SEQ ID NO: 12) | |
| (juxtamembrane | | |
| domain) | | |
| Transcription | Ga14-VP64 | |
| factor | | |

TABLE 7
Sequence component for protease activatable aEGFR CAR receptor

| Extracellular | Masking peptide | | QGQSGQCISPRGCPDGPYVMY |
|---|---|---|---|
| sensor | (to mask | | (SEQ ID NO: 4) |
| | αEGFR scFv) | | |
| | Linking | Flank linker | GSSGGSGGSGGSG |
| | peptide | | (SEQ ID NO: 29) |
| | | Protease | IEFDSG (SEQ ID NO: 5) or |
| | | cleavable | LVPRGSG (SEQ ID NO: 6) or |
| | | linker | PLGLAG (SEQ ID NO: 7) or |
| | | | Library of random |
| | | | amino acids |
| | | Flank linker | GSSGT (SEQ ID NO: 30) |

| | | |
|---|---|---|
| | αEGFR scFv | QILLTQSPVILSVSPGERVSFSC |
| | (derived from | RASQSIGTNIHWYQQRINGSPRL |
| | Cetuximab) | LIKYASESISGIPSRFSGSGSGT |
| | | DFTLSINSVESEDIADYYCQQNN |
| | | NWPTTFGAGTKLELKRGGGGSGG |
| | | GGSGGGGSQVQLKQSGPGLVQPS |
| | | QSLSITCTVSGFSLTNYGVHWVR |
| | | QSPGKGLEWLGVIWSGGNTDYNT |
| | | PFTSRLSINKDNSKSQVFFKMNS |
| | | LQSNDTAIYYCARALTYYDYEFA |
| | | YWGQGTLVTVSS (SEQ |
| | | ID NO: 32) |
| Transmembrane | CD8 hinge | TTTPAPRPPTPAPTIASQPLSLR |
| domain | | PEACRPAAGGAVHTRGLDFACDI |
| | | (SEQ ID NO: 33) |
| | CD28 TM + | FWVLVVVGGVLACYSLLVYVAF |
| | signaling | IIFWVRSKRSRGGHSDYMNMTP |
| | | RRPGPTRKHYQPYAPPRDFAAY |
| | | RS (SEQ ID NO: 34) |
| Intracellular | 4-1BB signaling | RFSVVKRGRKKLLYIFKQPFMR |
| domain | | PVQTTQEEDGCSCRFPEEEEGG |
| | | CEL (SEQ ID NO: 35) |
| | CD3z signaling | RVKFSRSADAPAYQQGQNQLYN |
| | | ELNLGRREEYDVLDKRRGRDPE |
| | | MGGKPRRKNPQEGLYNELQKDK |
| | | MAEAYSEIGMKGERRRGKGHDG |
| | | LYQGLSTATKDTYDALHMQALP |
| | | PR (SEQ ID NO: 36) |

### Example 4: T Cell Display for In Vitro Selection of Protease Substrates

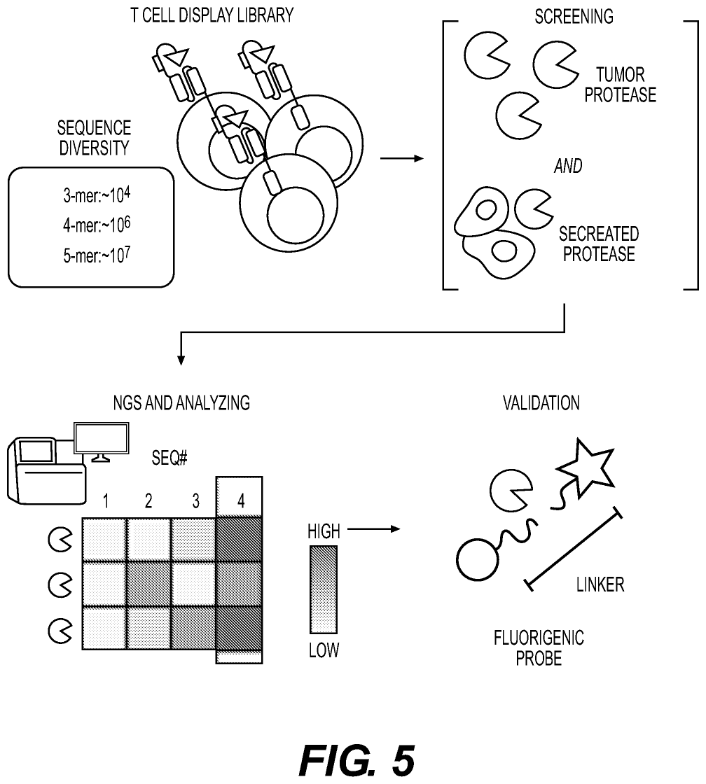

Synthetic peptide libraries are among the most widely used to screen substrates-based activity, however this technique is essentially limited by the size of libraries, which cannot exceed 10⁴ different amino acid sequences. Therefore, random substrate libraries of peptides of more than three amino acids are not complete. Larger library sizes (up to millions amino acid sequence diversity) are synthesized and investigated for screening protease substrates of T cell display in vitro with tumor-associated protease expressing in breast cancer (FIG. 5). Masked synNotch was constructed that genetically encodes libraries of protease-cleavable linkers. Then, protease substrates were screened against selected tumor-associated proteases and conditioned media from breast cancer cells. The sequences were then investigated from the cleavage activity by fluorogenic assays.

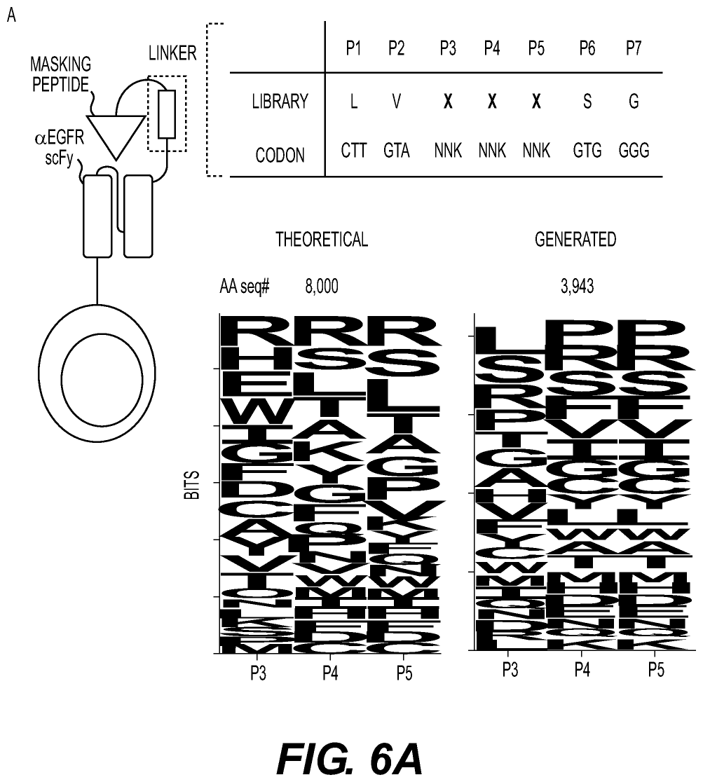

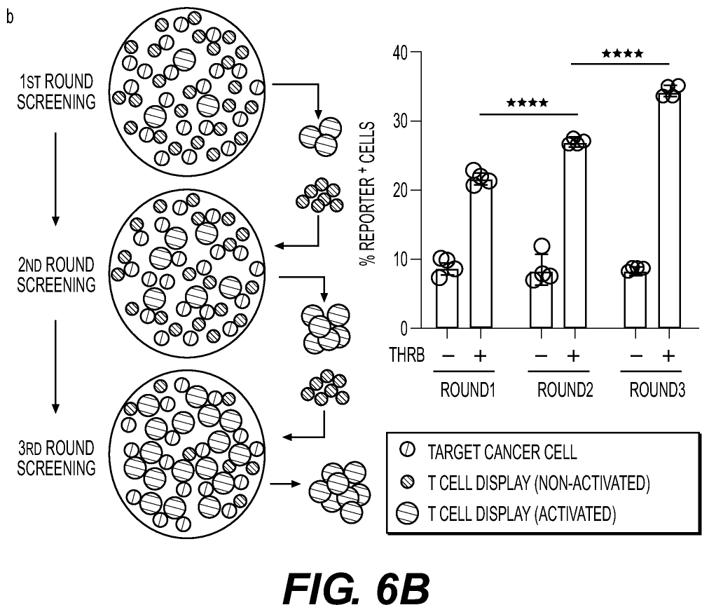

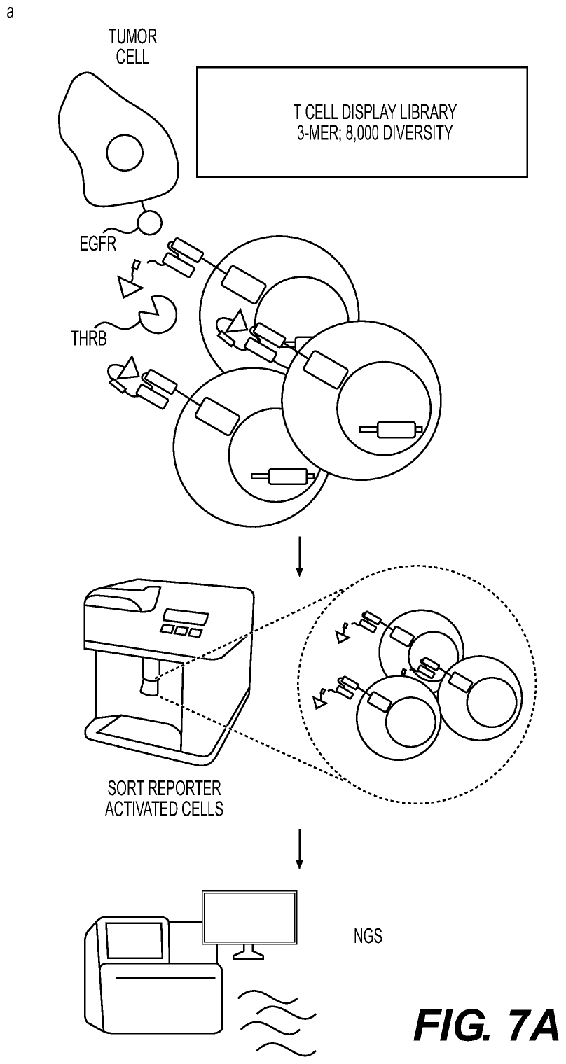

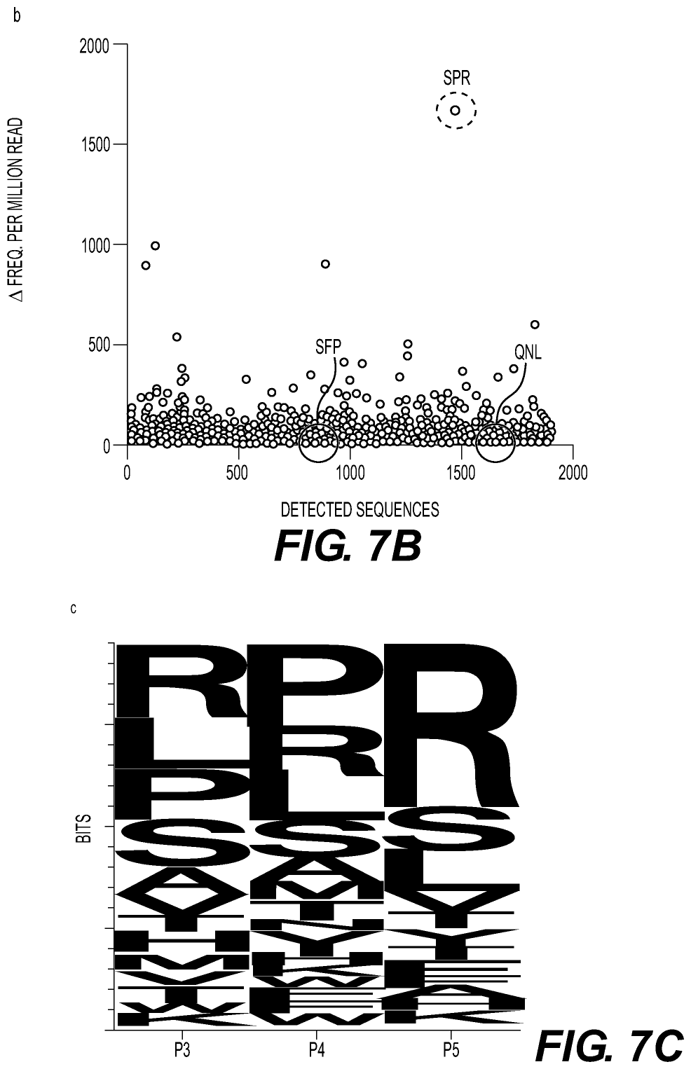

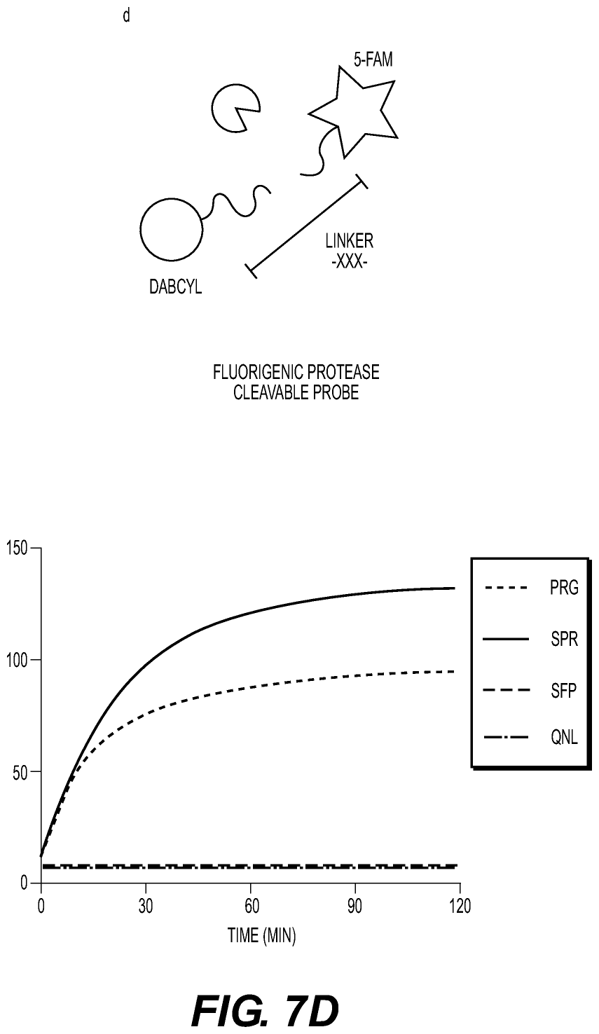

T cell display library for in vitro screening against Thrb protease. To illustrate the ability of masked T cell display for screening protease substrates in vitro, an anti-epidermal growth factor receptor (αEGFR) masked CAR Jurkat cell was constructed with a masked peptide mimotope (QGQSGQCISPRGCPDGPYVMY) (SEQ ID NO: 4) that genetically encodes libraries of 3-mer linker (LVXXXSG (SEQ ID NO: 19); X any 20 amino acids) to create an expected library size of 8,000 amino acid sequences (FIG. 6a). This library was cloned into a lentiviral vector. Lentivirus was tittered and transduced into T cells at an MOI favoring one insertion event per cell to express a single peptide substrate expression on each T cell display. At least 10× representation of codon library size was kept during cloning and transduction to keep library diversity. The quality and diversity were investigated by NGS. Similar consensus sequence motifs were identified from the generated library and compared to theoretical ones with 3,943 amino acid diversity sequences (FIG. 6a). The T cell display library was co-incubated with antigen-expressing target cells, MDA-MB-231 endogenously expressing EGFR, and Thrb protease. A significant increase in reporter activation was observed upon proteolysis by the Thrb and subsequent binding to target antigen cell (FIG. 6b). Multiple iterative cycles of screening and sorting of activated reporter cells demonstrated an increase in specific substrate in a large library. After each round of positive selection, significant increase in reporter activated cell was observed which shows the enrichment of Thrb specific peptide substrate.
In vitro screening by protease-activated T cell display could select the candidate Thrb substrate with high specificity. The library generated in FIG. 6 was then screened against Thrb and co-incubated with antigen-expressing target cells. For the round of screening, reporter activated cells were sorted and protease substrate sequences identified by NGS (FIG. 7a). The read count frequency of all unique sequences found in the screening were analyzed and evaluated by Δ frequency by comparing the read count frequency of treated and untreated Thrb protease conditions. (FIG. 7b). Consensus sequence motifs were identified by a position specific scoring matrix (PSSM) method which show consistency with the consensus substrate of thrombin (XPR|SX; MEROPS database) (FIG. 7c). The top positive of Δ frequency sequences (LVSPRSG (SEQ ID NO: 20)) and the top of negative Δ frequency sequences (LVSFPSG (SEQ ID NO: 21) and LVQNLSG (SEQ ID NO: 22)) were selected to investigate the cleavage activity by fluorogenic assays (FIG. 7d). These substrate sequences were synthesized with each peptide containing a quencher fluorophore pair at the termini (e.g., 5-FAM/Dabcyl) and then incubated with Thrb. The top hit sequences (LVSPRSG (SEQ ID NO: 20)) showed higher cleavage activity than the original Thrb cleavable sequence design (LVPRGSG (SEQ ID NO: 23)).
Peptide library encoded masked synNotch T cell display. T cell display was built with randomized libraries of protease-sensitive linker with different library size including 3-mer, 4-mer, 5-mer, and 6-mer using synthetic DNA containing NNK degenerate codons (where N=A/G/C/T and K=T/G) encoding all 20 amino acids at each of X position residues (Table 8). Degenerate oligonucleotides library was synthesized, and directly ligated into the gene site of the masked synNotch-containing lentiviral transfer plasmid. Plasmid ligation products were transformed into bacteria then amplified and used to generate lentivirus. Lentivirus were tittered and transduced into primary human T cells at an MOI favoring one insertion event per cell to produce a single substrate expression on each T cell display. The gDNA of T cell display libraries were extracted and investigated for the quality and diversity of the libraries by NGS (2×150 bps paired end reads on Ilumina MiSeq). Sequence filtering and peptide analysis was performed using an in-house informatics pipeline written in R. Some quality filters were applied: i) all the sequences with a stop codon were removed; ii) only the sequences that were in-frame were kept; iii) the bowtie 2 alignment mapping quality should be greater than 10. The codon frequency at each position and throughout the 4 amino acid windows should be uniform and match the expected input synthetic DNA. The limit of detection of each library is determined by mixing known substrates with libraries at different ratios (ranging from 1:10 to 1:100,000) and quantifying enrichment of the known sequences by NGS.

TABLE 8
Randomized library of protease-sensitive linker

| Linker | Codon diversity | Peptide diversity |
|---|---|---|
| 3-mer | 3.2 × 10⁴ | 8.0 × 10³ |
| 4-mer | 1.0 × 10⁶ | 1.6 × 10⁵ |
| 5-mer | 3.4 × 10⁷ | 3.2 × 10⁶ |
| 6-mer | 1.1 × 10⁹ | 6.4 × 10⁷ |

In vitro screening protease substrates with tumor-associated recombinant protease. The library established above is screened for tumor associated recombinant proteases by incubation in HER2 antigen coated wells. The target proteases include proteases corresponding to tumor microenvironment secretion. This includes members of the MMP family (MMP2, MMP8, MMP9 and MMP10) and fibroblast activation protein (FAP), which are secreted by majority of cancer cells and activate fibroblasts, as well as key members of the caspase (CASP3, CASP7, CASP8, CASP9) family that trigger cell death pathways upon protease activation. Negative selection is also screened using proteases that are found at high levels in circulating blood (e.g., thrombin, plasmin, C1s) to quantify potential background activity. Protease concentration is varied to investigate reporter activation level for the activity screening. Protease inhibitors are used to demonstrate protease-dependent reporter activation including N-Ethylmaleimide (caspase inhibitor), dichloroisocoumarin (FAP inhibitor) and ethylenediaminetetraacetic acid (MMP inhibitor). Multiple iterative cycles of screening and sorting of activated T cell display based on BFP expression demonstrated increased enrichment with each of three rounds of the selection. The sorted cells are to be cultured and at least 10× representation of sorted library cells are collected and prepared as an input sample for each screen. The gDNA of sorted cells are extracted to identify protease substrate sequences by NGS. A position specific scoring matrix (PSSM) method is deployed to identify consensus sequence motifs for the proteases. Consensus cleavage motifs are generated through multiple sequence alignments, based on which a PSSM is derived for high confidence prediction of precise cleavage sites within each selected sequence. The obtained sequences are compared with previously published substrate sequences, or sequences reported on MEROPS database. In vitro screening of protease substrates with tumor secreted protease. The library established above is screened for tumor secreted proteases from the coculture with HER2 expressing MDA-MB-468 and E0771 breast cancer cells. The T cell library is incubated with different effect to target ratio including 1:1, 1:5, 1:10 and 1:20 ratio and with varied co-incubation times including 24, 48 and 72 h. To test protease sensing specificity, commercially available protease inhibitor cocktail included serine protease (dichloroisocoumarin), metalloprotease (dichloroisocoumarin), cystine protease (N-Ethylmaleimide) and aspartate protease inhibitor (pepstatin) is used. The activated reporter cells are sorted based on BFP expression and gDNA of sorted cells is extracted to identify protease substrate sequences by NGS. A position specific scoring matrix (PSSM) method is also deployed to identify consensus sequence motifs for the proteases. A top list of substrates is nominated from the screening. These substrate sequences are synthesized with each peptide containing a quencher fluorophore pair at the termini (e.g., 5-FAM/Dabcyl). These substrates are evaluated by quantifying catalytic cleavage efficiencies (k_{cat}/K_{M}) using cancer cell culture supernatants and fluorescent analysis. The specificity of substrates is determined by assessing cross cleavage among proteases and off-target cleavage by serum proteases (e [symbol] complement and coagulation proteases). The mean fluorescent signal of triplicate samples is compared between proteases using a one-way ANOVA test (p<0.05).

### Example 5: T Cell Display for Protease Substrate Discovery in Mouse Models of Breast Cancer

Due to the coexistence of a large number of proteases in tumor which include cancer and stromal cell proteases, and their diverse roles in many biological pathways, it is difficult to identify specific cleavage products in a pool of complex cellular components. The objective in this example is to screen protease substrate by the T cell display technology in in vivo mouse models to determine whether it can discriminate on-from off-target protease activity leading to better substrate with high selectivity and specificity (FIG. 8).

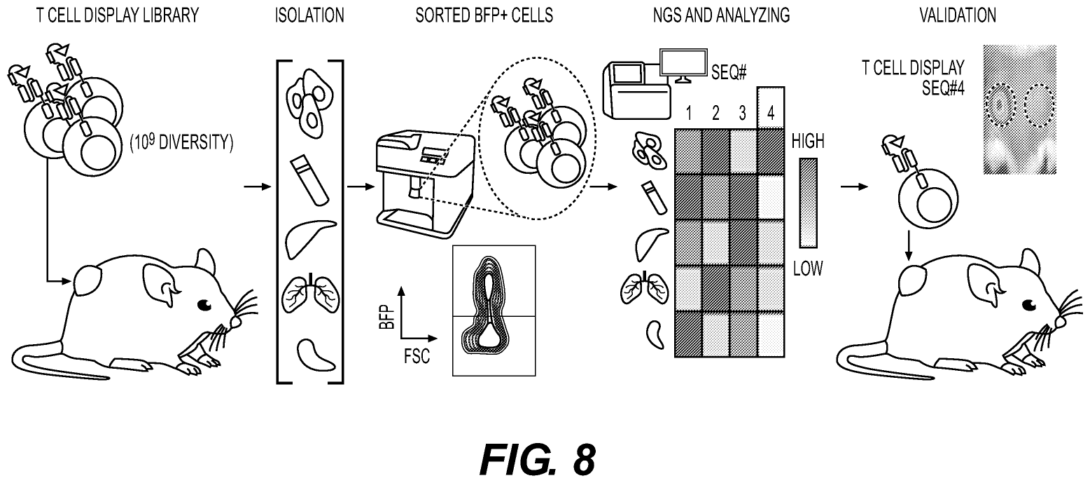

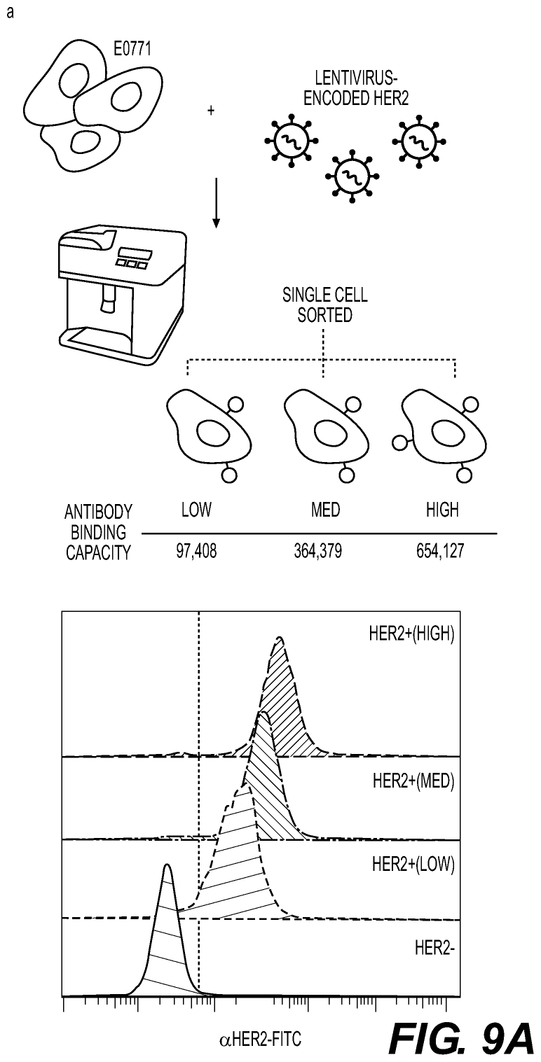

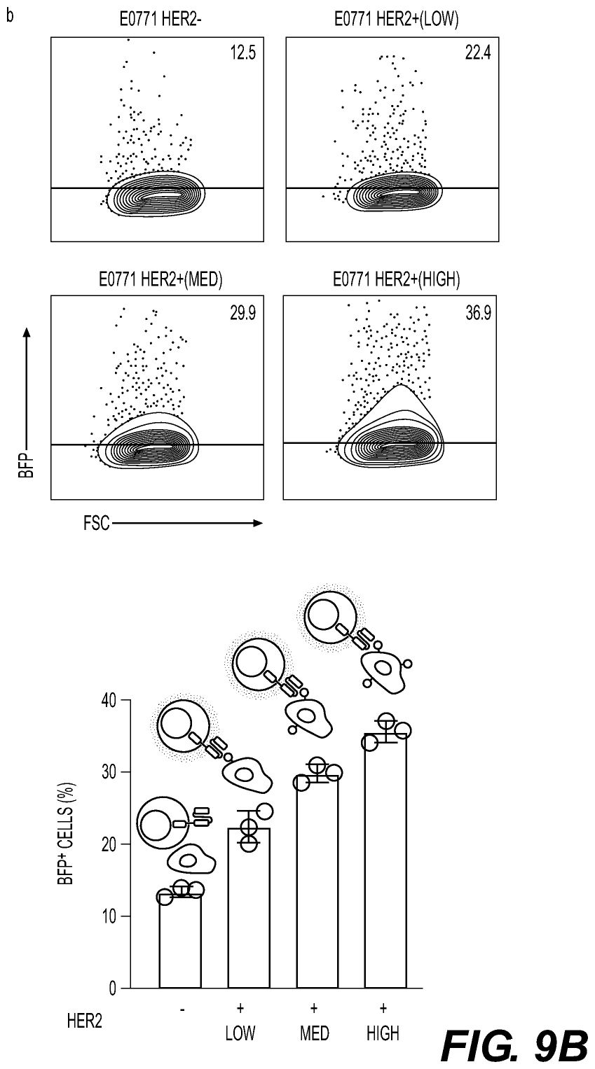

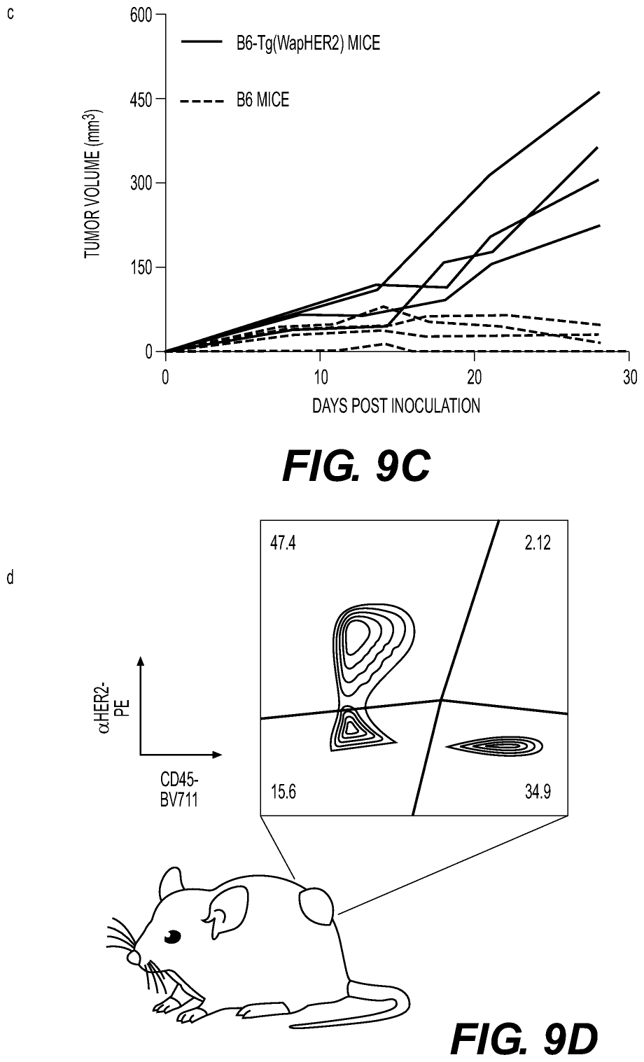

Established murine E0771 breast cancer cells expressing human HER2 antigen. To develop preclinical mouse models, C57BL/6-Tg (WapHER2) transgenic mice were used as hosts for syngeneic E0771 tumors expressing human HER2 and as recipients of murine T cells. First, lentivirus encoded human HER2 antigen was transduced into E0771 breast cancer cells. The transduced cells were then sorted into single cells on a 96-well plate using FACS Aria Fusion and cultured for 2-3 weeks. The different HER2 expression level of each clone was quantified cell surface antigen molecules in antibody binding capacity by Quantum Simply Cellular™ Microbead kit (FIG. 9a) and separated into low, medium, and high expression level. To assess whether Her2 antigen expressing on E0771 cells bind and activate αHER2 synNotch, the cancer cells were cocultured with synNotch and BFP reporter expression measured. The BFP expression increased with an increase of HER2 expression density on E0771 (FIG. 9b). Then, tumor growth kinetics were demonstrated in vivo by transplantation of HER2-expressing mouse E0771 cells (high expression level) into C57BL/6-Tg (WapHER2) transgenic mice. Due to tolerance to human HER2-associated antigen in this transgenic mouse, the tumors grew and expressed the antigen (FIGS. 9c and 9d).

In vivo masked synNotch reporter activation with potential positive and negative screening. To illustrate the ability of T cell display in discrimination of on-from off-target protease activity, 10 million of αHer2 masked synNotch T cells library (4-mer linker) was intravenously injected into healthy and MDA-MB-468 HER2+ tumor bearing (subcutaneously xenograft) NSG mice. After adoptive transfer for 24 h, the T cells were isolated from the tumor and others organ including blood, spleen, liver, lung, to analyze the increase BFP expression level. Upon T cell displays infiltrating into tumors, the peptide linkers were cleaved by tumor associated proteases leading to induction of synNotch activation and downstream BFP expression (FIG. 10a). Significantly higher percent of BFP+ T display cells were found in tumors compared to ones in other organs of both healthy and tumor bearing mice (FIGS. 10b and 10c). These data show that masked synNotch T cell display selectively activated in a protease-activated tumor and were capable to be utilized for discrimination of on-from off-target activity in vivo.

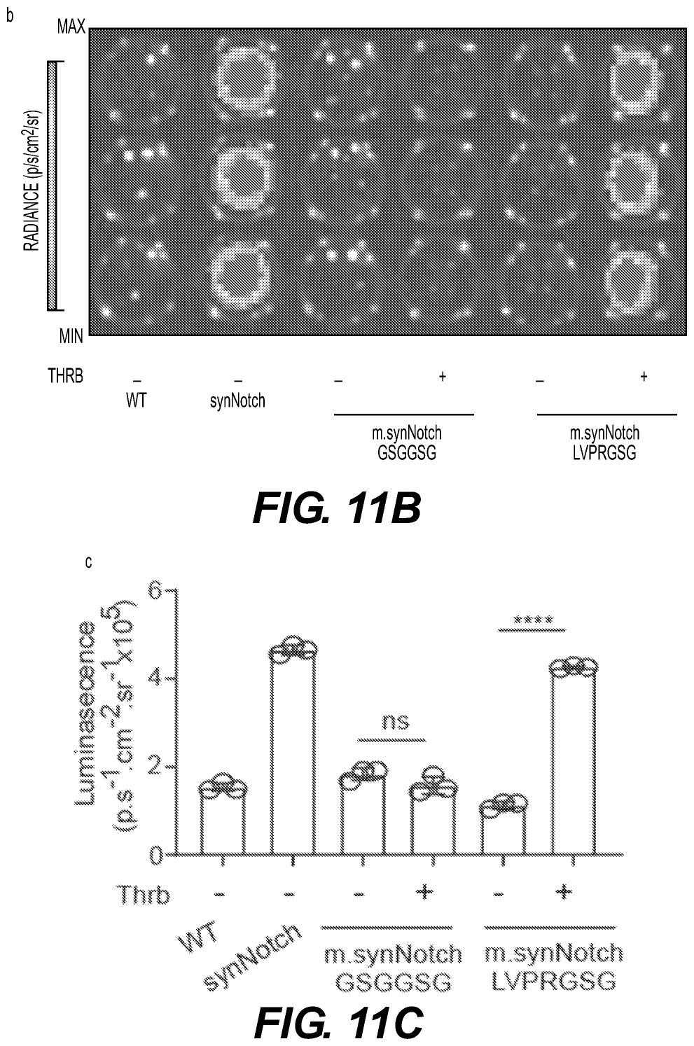

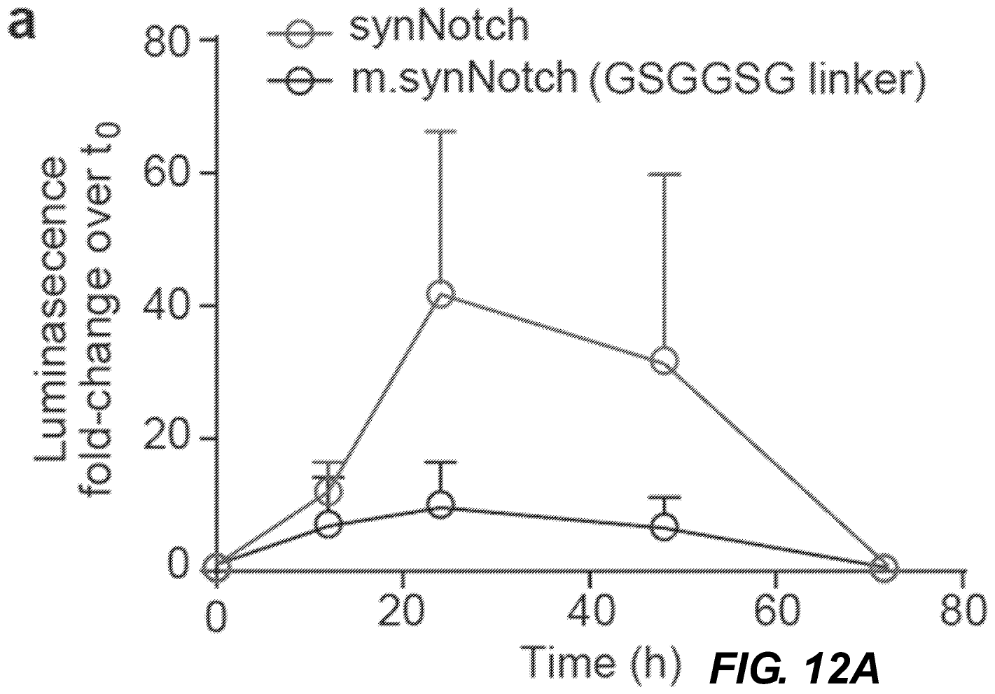

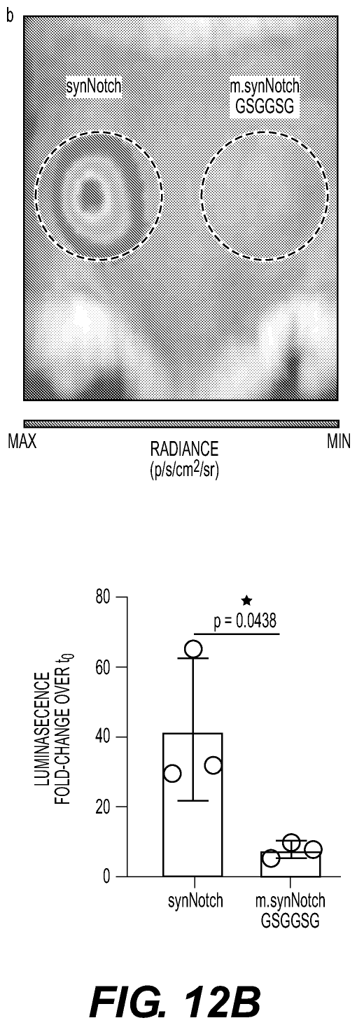

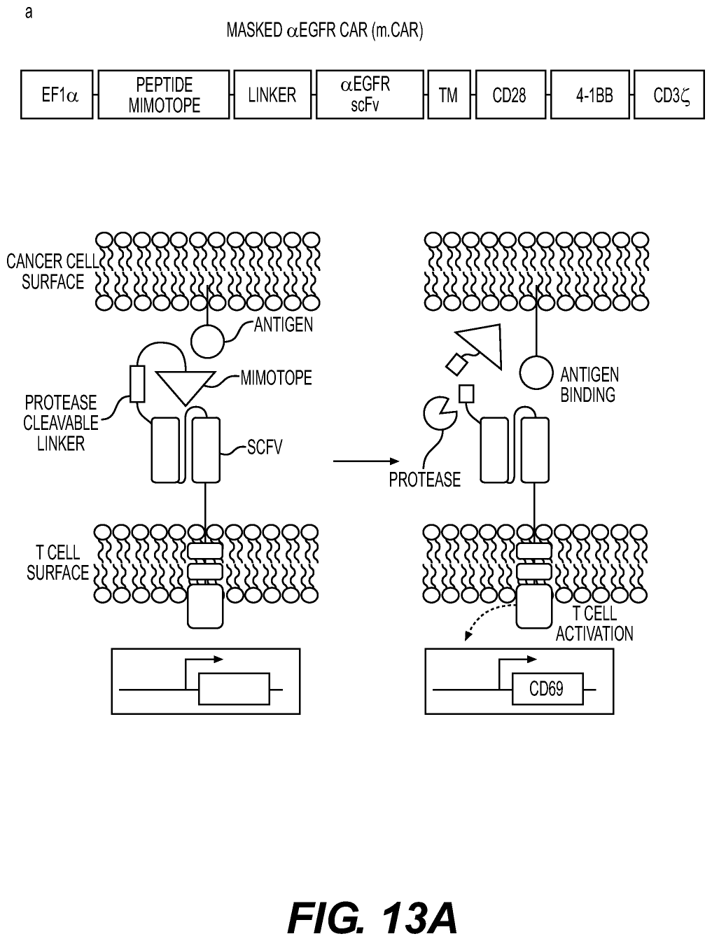

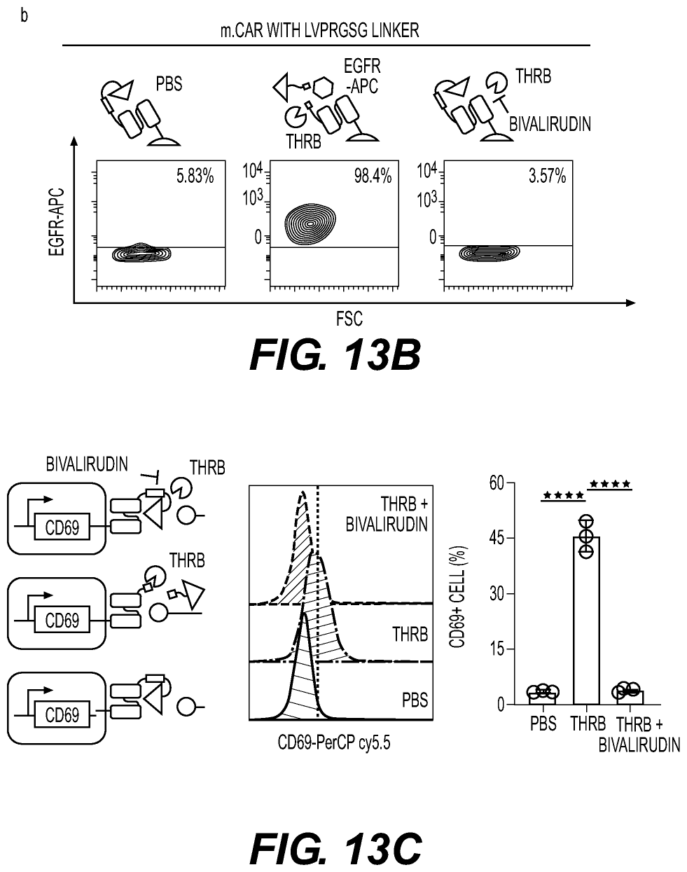

Luciferase reporter for detection of synNotch activation in vivo. T cells engineered with synNotch receptors have robust and highly controlled custom behaviors. To investigate the feasibility of in vivo imaging of protease activity in mouse model, the Gal4 transcriptional response elements were first cloned to co-expresses a destabilized copepod GFP and Firefly (Fluc) luciferase (FIG. 11a). Then, this reporter gene was co-transduced with αHER2 masked synNotch receptor gene into primary human T cells. Masked synNotchs with luciferase reporter were co-cultured with HER2+ breast cancer line MDA-MB-468 for 24 h with or without treated protease. The level of luciferase expression upon synNotch activation was measured by an IVIS Spectrum CT system after adding luciferin to the media. Similar to BFP reporter gene, masked synNotchs with LVPRGSG (SEQ ID NO: 23) linker induced synNotch activation and downstream high luciferase luminescence expression as same as ones in unmasked synNotch upon proteolysis by Thrb and stimulation by MDA-MB-468 HER2⁺ cells (FIG. 11b and c). Then, the luciferase reporter was utilized to detect the synNotch activation in vivo. NSG mice with bilateral flank tumors are inoculated with MDA-MB-468 HER2+ tumor. Engineered T cells with luciferase reporter into both sites of tumor were intratumorally transferred with one cohort receiving αHER2 synNotch (unmasked) cells and a separate cohort receiving αHER2 masked synNotch cells with GSGGSG (SEQ ID NO: 15) linker (negative control) to model activated and nonactivated T cell display, respectively. At 24 h after adoptive transfer luminescence was significantly increased in the tumors receiving αHER2 synNotch compared with the tumors receiving αHER2 masked synNotch cells with GSGGSG linker in the same animal (FIG. 12).

In vivo screening of protease substrate activity in human breast cancer xenografts in mice. Protease substrates in HER2 expressing MDA-MB-468 tumors are screened with T cell display libraries above. First, tumor growth kinetics and biodistribution of the T cell display are characterized by inoculating one million of MDA-MB-468 cells in the mammary fat pads. After tumors reach ˜100 mm³, T cell display encoded library is intravenously injected (firefly luciferase reporter) (10, 20, and 50 million cells) into tumor bearing mice and naïve mice as a control. After T cell transfer for 24, 48, and 72 hours, migration is tracked by luminescence images acquired by an IVIS Spectrum CT system and then harvested the T cells from tumor, lung, spleen, liver and collect blood to analyze the cell number in each organ. T cell injection dose is selected from the minimum does that provide maximum T cell accumulated in the tumor. In addition, the time point selected provides the maximum reporter+ T cell number in the tumors. The number of engineered T cells in the tumor is important to determine the peptide library and injected T cell number. The number of T cells migrating tumor should be at least 2-5 times bigger than the peptide substrate library size to maintain diversity for screening. For screening, the T cell display library is injected to healthy and tumor bearing mice. After the selected time point, protease-activated tumor-infiltrating T cells with BFP reporter expression are sorted (BD FacsAria II). gDNA is extracted from sorted cells and primers are designed to anneal to regions flanking the library gene site on the viral transgene and are directly tailed with Illumina adapted primers containing population-specific index sequences. Sequencing is performed on the Illumina MiSeq platform ((2×150 bps paired-end reads). For each data set, a frequency rank ordered list of protease substrate is identified and found in the tumor but not in healthy organs. Then, a position specific scoring matrix method is deployed to identify consensus sequence motifs for the protease substrate signature.

In vivo screening of protease substrate signature in mouse breast cancer in C57BL/6-Tg (WapHER2) transgenic mice. As NSG mice lack an intact immune system, this technology was further evaluated in C57BL/6-Tg (WapHER2) transgenic mice as hosts for syngeneic E0771 tumors expressing HER2 and as recipients of murine T cells. This transgenic mouse line displays tolerance to HER2 antigen which is necessary for establishing the murine model of breast cancer expressing HER2. These mice are breed in-house as hemizygotes, and also bred with B6 mice to establish B6-HER2 hemizygotes. The murine masked synNotch T cell display is screened for protease substrate as above. The activated T cells are sorted and the gDNA extracted and then the hit sequence is recovered by NGS.

Substrate signatures associated with breast cancer. Based on the lists of protease substrates above, a set of genetically encoded hit substrates are cloned into masked synNotch cells with firefly luciferase reporter as established. These set of masked synNotch T cells are intravenously injected to tumor bearing mice (in separate cohort of each substrate-encoded T cell) and are monitored for luciferase signal with whole mouse body through an IVIS Spectrum CT. At a selected time point (determined above), the tumor and other organs including lung, liver, spleen, kidney, lymph nodes are harvested and the luciferase signal is measured. The median luciferase signal of biologic replicates is compared between groups using a Mann Whitney Test (p<0.05) to determine the set of protease substrate signatures.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

1. An engineered protease activatable receptor comprising an extracellular sensor, a transmembrane domain, and an intracellular domain wherein the extracellular sensor further comprises a masking peptide, a linking peptide, an antigen binding receptor, and a signal transducing complex.

2. The engineered protease activatable receptor of claim 1, wherein the antigen binding receptor is an extracellular surface receptor; and/or wherein the engineered receptor comprises a synthetic Notch (synNotch) receptor or a chimeric antigen receptor (CAR).

3. (canceled)

4. The engineered protease activatable receptor of claim 1, wherein the antigen binding receptor is an anti-human epidermal growth factor receptor 2 (αHER2) scFv receptor comprising SEQ ID NO: 27 or an anti-epidermal growth factor receptor (αEGFR) scFv receptor.

5. (canceled)

6. The engineered protease activatable receptor of claim 1, wherein the masking peptide is 9-21 amino acids in length.

7. The engineered protease activatable receptor of claim 1, wherein the masking peptide is 9 amino acids in length, or 10 amino acids in length, or 11 amino acids in length, or 12 amino acids in length.

8. (canceled)

9. (canceled)

10. (canceled)

11. (canceled)

12. (canceled)

13. The engineered protease activatable receptor of claim 1, wherein the linking peptide is 5-40 amino acids in length.

14. The engineered protease activatable receptor of claim 1, wherein the linking peptide is 6 amino acids in length, or 7 amino acids in length, or 8 amino acids in length, or 9 amino acids in length.

15. (canceled)

16. (canceled)

17. (canceled)

18. The engineered protease activatable receptor of claim 1, wherein the masking peptide comprises SEQ ID NO: 1-4, a αHER2 peptide, or an αEGFR peptide; wherein the linking peptide comprises a protease cleavable peptide; wherein the signal transducing complex comprises a CD8α signaling peptide, an extracellular Notch core, and a juxtamembrane Notch core; and/or wherein the transmembrane domain is a transmembrane Notch core.

19. The engineered protease activatable receptor of claim 18, wherein the protease cleavable peptide comprises SEQ ID NO: 5-7.

20. (canceled)

21. The engineered protease activatable receptor of claim 18, wherein the CD8α signaling peptide comprises SEQ ID NO: 8 or SEQ ID NO: 9; wherein the extracellular Notch core comprises SEQ ID NO: 10 or SEQ ID NO: 11; and/or wherein the juxtamembrane Notch core comprises SEQ ID NO: 12.

22. (canceled)

23. (canceled)

24. (canceled)

25. The engineered protease activatable receptor of claim 18, wherein the transmembrane Notch core comprises SEQ ID NO: 13 or SEQ ID NO: 14.

26. The engineered protease activatable receptor of claim 1, wherein the intracellular transcription factor activates a reporter gene.

27. The engineered protease activatable receptor of claim 26, wherein the transcription factor comprises a Gal4-VP64 transcription factor comprising SEQ ID NO: 28; and/or wherein the reporter gene comprises CD69, luciferase, green fluorescent protein (GFP), yellow fluorescent protein (YFP), blue fluorescent protein (BFP), cyan fluorescent protein (CFP), monomeric red fluorescent protein (mRFP), Discosoma striata (DsRed), mCherry, mOrange, tdTomato, mStrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, EBFP2, Herpes simplex virus thymidine kinase (HSV-TK), and/or sodium iodide symporter (NIS).

28. (canceled)

29. An immune cell comprising the engineered protease activatable receptor of claim 1.

30. (canceled)

31. The immune cell of claim 29, further comprising a library of protease cleavable substrates in a linking peptide domain, wherein the library comprises a random 3, 4, 5, or 6 amino acid peptide.

32. (canceled)

33. An in vitro method of screening an assortment of protease substrates for monitoring dysregulated protease enzymes in a cancer comprising
a. culturing a cancerous sample expressing a target antigen with i) an immune cell comprising an extracellular sensor, a transmembrane domain, and an intracellular domain wherein the extracellular sensor further comprises a masking peptide, a library of protease substrates, an antigen binding receptor, and a signal transducing complex and ii) a protease; wherein a substrate that is a specific target for the protease is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; and
b. detecting expression of a reporter gene

34. The method of claim 33, further comprising one or both of: (i) identifying the sequence of the protease substrate by sequencing; and (ii) successively performing the screen at least 2-5 times and each subsequent time only using the top 10% of sequences from the previous round.

35. (canceled)

36. (canceled)

37. The method of claim 33, wherein any one of the protease substrates comprises a random 3 amino acid in a linker peptide, a random 4 amino acid in the linker peptide, a random 5 amino acid in the linker peptide, or a random 6 amino acid in the linker peptide.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. An in vivo method of screening of protease substrates for monitoring dysregulated protease enzymes in a cancer comprising step of:
a. obtaining an engineered immune cell comprising a protease activatable receptor (CAR or synNotch receptor) which comprises an extracellular sensor, a transmembrane domain, and an intracellular domain; wherein the extracellular sensor comprises a masking peptide, a library of protease substrates and an antigen binding receptor;
b. transferring the engineered immune cells to tumor bearing mice expressing target antigen; wherein the tumor microenvironment is protease enriched; wherein a substrate that is a specific target for the proteases is cleaved by the protease; and wherein cleavage of the protease substrate causes removal of the masking peptide thereby allowing the antigen binding receptor to bind to the target antigen on the cancerous sample; wherein binding of the antigen binding receptor to the target antigen causes the signal transducing complex to express the reporter gene; wherein binding of the target further causes tumor tissue to be dissociated; and
c. detecting expression of the reporter gene.

51. The method of claim 50, further comprising one or more of: (i) sorting for the expression of the reporter gene; (ii) detecting and sorting cells expressing the reporter gene in blood or healthy tissues for negative selection; and (iii) identifying the sequence of the protease by sequencing.

52. (canceled)

53. (canceled)

54. (canceled)
